(19) United States
(12) Patent Application Publication
(10) Pub. No.: US 2018/0201910 A1
(43) Pub. Date: Jul. 19, 2018

(54) RECOMBINANT MICROORGANISM CAPABLE OF SYNTHESIZING ALKANE AND METHOD FOR PRODUCING ALKANE

(71) Applicant: TOYOTA JIDOSHA KABUSHIKI KAISHA, Toyota-shi (JP)

(72) Inventors: Masayoshi MURAMATSU, Miyoshi-shi (JP); Shusei OBATA, Nagoya-shi (JP)

(73) Assignee: TOYOTA JIDOSHA KABUSHIKI KAISHA, Toyota-shi (JP)

(21) Appl. No.: 15/867,922

(22) Filed: Jan. 11, 2018

(30) Foreign Application Priority Data
Jan. 17, 2017 (JP) 2017-006246

(51) Int. Cl. C12N 9/02; C12P 5/02; C12N 9/88; C07K 14/405; C12N 15/70

(52) U.S. Cl. CPC C12N 9/0008; C12P 5/02; C12N 15/70; C12N 9/88; C07K 14/405; C12Y 102/0108

(57) ABSTRACT

The capacity for alkane synthesis is to be significantly improved with the use of an acyl-ACP reductase gene and a decarbonylase gene derived from blue-green algae. A recombinant microorganism comprising, as foreign genes, an acyl-ACP reductase gene and a decarbonylase gene derived from blue-green algae comprises 4 types of ferredoxin genes and 3 types of ferredoxin reductase genes derived from blue-green algae in predetermined combinations.

The present application claims priority from Japanese patent application JP 2017-006246 filed on Jan. 17, 2017, the content of which is hereby incorporated by reference into this application.

### BACKGROUND

### Field

The present disclosure relates to a recombinant microorganism capable of synthesizing alkane that can be used for a biodiesel fuel etc. and a method for producing alkane using the same.

### Description of Related Art

Alkane contained in petroleum is used for various applications after purification via fractional distillation. Also, alkane is not only used widely as a raw material in chemical industry, but is also used as a major ingredient of a diesel fuel obtained from petroleum. In recent years, a technique that allows coexpression of an acyl-ACP reductase gene and a decarbonylase gene derived from blue-green algae in E. coli to produce an alkane as a light oil component via fermentation was developed (U.S. Pat. No. 8,846,371).

It is also reported that a decarbonylase, which is a key enzyme for alkane synthesis, requires ferredoxin and ferredoxin reductase (Science, Vol. 329, p. 559-562, 2010) and that alkane synthesis in Saccharomyces cerevisiae requires coexpression of the ferredoxin gene and the ferredoxin reductase gene derived from E. coli, in addition to the decarbonylase gene (Biotechnology Bioengineering, Vol. 112, No. 6, p. 1275-1279, 2015). According to Biotechnology Bioengineering, Vol. 112, No. 6, p. 1275-1279, 2015, however, the amount of alkane production is approximately 3 jag/g-dry cells. In such a case, Saccharomyces cerevisiae exhibits OD600 of approximately 20 at full growth, and the dry cell weight is approximately 4 g-dry cells/l. In accordance with the method described in Biotechnology Bioengineering, Vol. 112, No. 6, p. 1275-1279, 2015, the amount of production would be as low as approximately 12 μg/l.

In addition, WO 2013/024527 discloses that, for example, the Saccharomyces cerevisiae-derived ferredoxin gene and the Saccharomyces cerevisiae-derived ferredoxin reductase may be introduced into a recombinant yeast that has acquired the capacity of synthesizing alkane (i.e., recombinant Saccharomyces cerevisiae), so that the capacity of alkane synthesis may be improved. Also, WO 2013/024527 shows the amount of alkane production attained when the ferredoxin gene and the ferredoxin reductase gene derived from E. coli are subjected to coexpression with acyl-ACP reductase and decarbonylase in E. coli in FIG. 10. In WO 2013/024527, the amount of alkane production attained without the introduction of ferredoxin and ferredoxin reductase is designated as 100, and the amount of alkane production attained with the introduction thereof would be 100 or less. That is, alkane productivity is not enhanced through coexpression of ferredoxin and ferredoxin reductase.

### SUMMARY

Under the above circumstances, the present disclosure provides a recombinant microorganism that has the excellent capacity for alkane synthesis with the use of an acyl-ACP reductase gene and a decarbonylase gene derived from blue-green algae and a method for producing alkane with excellent productivity with the use of such recombinant microorganism.

In an alkane synthesis reaction system involving the use of an acyl-ACP reductase gene and a decarbonylase gene derived from blue-green algae, predetermined pairs of genes selected from among a plurality of ferredoxin genes and a plurality of ferredoxin reductase genes derived from blue-green algae may be used. Thus, the capacity for alkane synthesis can be improved to a significant extent. Accordingly, the present disclosure is as described below.

Specifically, the present disclosure is as follows.

(1) A recombinant microorganism comprising, as foreign genes, an acyl-ACP reductase gene and a decarbonylase gene derived from blue-green algae,

provided that 4 types of blue-green algae-derived ferredoxin genes are designated as the FD gene 1, the FD gene 2, the FD gene 3, and the FD gene 4 and 3 types of blue-green algae-derived ferredoxin reductase genes are designated as the FDR gene 1, the FDR gene 2, and the FDR gene 3, the recombinant microorganism capable of synthesizing alkane comprises a ferredoxin gene and a ferredoxin reductase gene in the combination as described below:

the FD gene 1 in combination with the FDR gene 1;

the FD gene 1 in combination with the FDR gene 2;

the FD gene 2 in combination with the FDR gene 1;

the FD gene 2 in combination with the FDR gene 2;

the FD gene 2 in combination with the FDR gene 3;

the FD gene 3 in combination with the FDR gene 2

the FD gene 3 in combination with the FDR gene 3;

the FD gene 4 in combination with the FDR gene 1; or

the FD gene 4 in combination with the FDR gene 2,

wherein the FD gene 1 encodes a protein [a1] or [b1]:

[a1] a protein comprising the amino acid sequence as shown in SEQ ID NO: 2; or

[b1] a protein comprising an amino acid sequence having identity of higher than 61.2% to the amino acid sequence as shown in SEQ ID NO: 2 and functioning as ferredoxin,

wherein the FD gene 2 encodes a protein [a2] or [b2]:

[a2] a protein comprising the amino acid sequence as shown in SEQ ID NO: 4; or

[b2] a protein comprising an amino acid sequence having identity of higher than 60% to the amino acid sequence as shown in SEQ ID NO: 4 and functioning as ferredoxin,

wherein the FD gene 3 encodes a protein [a3] or [b3]:

[a3] a protein comprising the amino acid sequence as shown in SEQ ID NO: 6; or

[b3] a protein comprising an amino acid sequence having identity of higher than 62.9% to the amino acid sequence as shown in SEQ ID NO: 6 and functioning as ferredoxin,

wherein the FD gene 4 encodes a protein [a4] or [b4]:

[a4] a protein comprising the amino acid sequence as shown in SEQ ID NO: 8; or

[b4] a protein comprising an amino acid sequence having identity of higher than 62.3% to the amino acid sequence as shown in SEQ ID NO: 8 and functioning as ferredoxin,

wherein the FDR gene 1 encodes a protein [c1] or [d1]:

[c1] a protein comprising the amino acid sequence as shown in SEQ ID NO: 10; or

[d1] a protein comprising an amino acid sequence having identity of 60% or higher to the amino acid sequence as shown in SEQ ID NO: 10 and functioning as ferredoxin reductase,

wherein the FDR gene 2 encodes a protein [c2] or [d2]:

[c2] a protein comprising the amino acid sequence as shown in SEQ ID NO: 12; or

[d2] a protein comprising an amino acid sequence having identity of 60% or higher to the amino acid sequence as shown in SEQ ID NO: 12 and functioning as ferredoxin reductase, and

wherein the FDR gene 3 encodes a protein [c3] or [d3]:

[c3] a protein comprising the amino acid sequence as shown in SEQ ID NO: 14; or

[d3] a protein comprising an amino acid sequence having identity of 60% or higher to the amino acid sequence as shown in SEQ ID NO: 14 and functioning as ferredoxin reductase.

(2) The recombinant microorganism according to (1), wherein the blue-green algae-derived decarbonylase gene encodes a protein [e] or [f]:

[e] a protein comprising the amino acid sequence as shown in SEQ ID NO: 16; or

[f] a protein comprising an amino acid sequence having identity of 60% or higher to the amino acid sequence as shown in SEQ ID NO: 16 and having decarbonylase activity.

(3) The recombinant microorganism according to (1), wherein the blue-green algae-derived acyl-ACP reductase gene encodes a protein [g] or [h]:

[g] a protein comprising the amino acid sequence as shown in SEQ ID NO: 18; or

[h] a protein comprising an amino acid sequence having identity of 60% or higher to the amino acid sequence as shown in SEQ ID NO: 18 and having acyl ACP reductase activity.

(4) The recombinant microorganism according to (1), wherein host cells are E. coli or Klebsiella bacteria.
(5) A method for producing alkane comprising a step of culturing the recombinant microorganism according to any of (1) to (4) above.
(6) The method for producing alkane according to (5), which further comprises a step of recovering alkane from a medium in which the recombinant microorganism is cultured.
(7) The method for producing alkane according to (5), which further comprises a step of recovering alkane from a medium in which the recombinant microorganism is cultured and purifying the recovered alkane.
(8) The method for producing alkane according to (5), wherein alkane having 9 to 20 carbon atoms is produced.

According to the present disclosure, it is possible to improve the capacity of a recombinant microorganism comprising, as foreign genes, an acyl-ACP reductase gene and a decarbonylase gene derived from blue-green algae for alkane synthesis to a significant extent.

In comparison with a conventional recombinant microorganism comprising, as foreign genes, an acyl-ACP reductase gene and a decarbonylase gene derived from blue-green algae, specifically, the recombinant microorganism according to the present disclosure has a superior capacity for alkane synthesis. With the use of the recombinant microorganism according to the present disclosure, alkane productivity can be improved to a significant extent in an alkane synthesis system involving the use of microorganisms. That is, a cost for alkane production can be reduced to a significant extent.

### BRIEF DESCRIPTIONS OF THE DRAWINGS

FIG. 1 shows the nucleotide sequence of an artificial gene designed through codon optimization of YP_001865390;

FIG. 2 shows the nucleotide sequence of an artificial gene designed through codon optimization of YP_001866231;

FIG. 3 shows the nucleotide sequence of an artificial gene designed through codon optimization of YP_001868825;

FIG. 4 shows the nucleotide sequence of an artificial gene designed through codon optimization of YP_001864061;

FIG. 5 shows the nucleotide sequence of an artificial gene designed through codon optimization of YP_001864105;

FIG. 6 shows the nucleotide sequence of an artificial gene designed through codon optimization of YP_001864826;

FIG. 7 shows the nucleotide sequence of an artificial gene designed through codon optimization of YP_001865513;

FIG. 8 shows the nucleotide sequence of an artificial gene designed through codon optimization of YP_001867060;

FIG. 9 shows the nucleotide sequence of an artificial gene designed through codon optimization of YP_001865513b;

FIG. 10 shows the nucleotide sequence of an artificial gene designed through codon optimization of YP_001866231b;

FIG. 11 shows a characteristic diagram demonstrating the results of assaying the amounts of alkane production of Strain Nos: 2 to 17 via GC/MS and representing the average values (2 days after the initiation of culture);

FIG. 12 shows a characteristic diagram demonstrating the results of assaying the amounts of alkane production of Strain Nos: 2 to 17 via GC/MS and representing the average values (3 days after the initiation of culture);

FIG. 13 shows a characteristic diagram demonstrating the results of assaying the amounts of alkane production of Strain Nos: 2 to 17 via GC/MS and representing the average total amount of alkane (2 days after the initiation of culture);

FIG. 14 shows a characteristic diagram demonstrating the results of assaying the amounts of alkane production of Strain Nos: 2 to 17 via GC/MS and representing the average total amount of alkane (3 days after the initiation of culture);

FIG. 15 shows a characteristic diagram demonstrating the results of assaying the amount of alkane production of the Klebsiella sp. 100048 strain into which FDR2 and Fd7 had been introduced in combination.

### DETAILED DESCRIPTION OF THE EMBODIMENTS

Hereafter, preferred embodiments are described in detail with reference to the drawings and examples.

The present disclosure relates to a recombinant microorganism comprising, as foreign genes, an acyl-ACP reductase gene and a decarbonylase gene derived from blue-green algae, and such recombinant microorganism comprises a ferredoxin gene and a ferredoxin reductase gene in a predetermined combination. A recombinant microorganism comprising a ferredoxin gene and a ferredoxin reductase gene in a predetermined combination has the capacity for alkane synthesis that is superior to that of a recombinant microorganism comprising a ferredoxin gene and a ferredoxin reductase gene in a different combination.

### <Combinations of Ferredoxin Gene and Ferredoxin Reductase Gene>

### [Ferredoxin Gene]

Ferredoxin is an iron-sulfur protein containing iron-sulfur clusters (Fe—S clusters) therewithin and functioning as an electron carrier. Ferredoxin genes that can be used in the predetermined combinations described above are 4 types of ferredoxin genes selected from among many ferredoxin genes possessed by blue-green algae. Such 4 types of ferredoxin genes are referred to as the FD gene 1, the FD gene 2, the FD gene 3, and the FD gene 4.

The FD gene 1 encodes a protein [a1] or [b1]:

[a1] a protein comprising the amino acid sequence as shown in SEQ ID NO: 2; or

[b1] a protein comprising an amino acid sequence having identity of higher than 61.2% to the amino acid sequence as shown in SEQ ID NO: 2 and functioning as ferredoxin.

The amino acid sequence as shown in SEQ ID NO: 2 is registered as the amino acid sequence of ferredoxin 2Fe-2S derived from Nostoc punctiforme PCC 73102 (Accession Number: YP_001864105 or (WP_012407188)). SEQ ID NO: 1 shows the nucleotide sequence encoding the amino acid sequence as shown in SEQ ID NO: 2.

The FD gene 2 encodes a protein [a2] or [b2]:

[a2] a protein comprising the amino acid sequence as shown in SEQ ID NO: 4; or

[b2] a protein comprising an amino acid sequence having identity of higher than 60% to the amino acid sequence as shown in SEQ ID NO: 4 and functioning as ferredoxin.

The amino acid sequence as shown in SEQ ID NO: 4 is registered as the amino acid sequence of ferredoxin 2Fe-2S derived from Nostoc punctiforme PCC 73102 (Accession Number: YP_001864826 (or WP_012407904)). SEQ ID NO: 3 shows the nucleotide sequence encoding the amino acid sequence as shown in SEQ ID NO: 4.

The FD gene 3 encodes a protein [a3] or [b3]:

[a3] a protein comprising the amino acid sequence as shown in SEQ ID NO: 6; or

[b3] a protein comprising an amino acid sequence having identity of higher than 62.9% to the amino acid sequence as shown in SEQ ID NO: 6 and functioning as ferredoxin.

The amino acid sequence as shown in SEQ ID NO: 6 is registered as the amino acid sequence of ferredoxin 2Fe-2S derived from Nostoc punctiforme PCC 73102 (Accession Number: YP_001865513 (or WP_012408585)). SEQ ID NO: 5 shows the nucleotide sequence encoding the amino acid sequence as shown in SEQ ID NO: 6.

The FD gene 4 encodes a protein [a4] or [b4]:

[a4] a protein comprising the amino acid sequence as shown in SEQ ID NO: 8; or

[b4] a protein comprising an amino acid sequence having identity of higher than 62.3% to the amino acid sequence as shown in SEQ ID NO: 8 and functioning as ferredoxin.

The amino acid sequence as shown in SEQ ID NO: 8 is registered as the amino acid sequence of ferredoxin 2Fe-2S derived from Nostoc punctiforme PCC 73102 (Accession Number: YP_001867060 (or WP_012410088)). SEQ ID NO: 7 shows the nucleotide sequence encoding the amino acid sequence as shown in SEQ ID NO: 8.

In addition to the 4 types of ferredoxin genes described above (i.e., the FD gene 1, the FD gene 2, the FD gene 3, and the FD gene 4), a gene encoding ferredoxin 4Fe-4S that is registered under Accession Number: YP_001864061 (or WP_012407144) (hereafter, referred to as the “FD gene 5”) is known as a ferredoxin gene derived from Nostoc punctiforme PCC 73102. The FD gene 5 encodes the amino acid sequence as shown in SEQ ID NO: 20. SEQ ID NO: 19 shows the nucleotide sequence encoding the amino acid sequence as shown in SEQ ID NO: 20.

Ferredoxin encoded by the FD gene 5 has identity of 61.2% to ferredoxin encoded by the FD gene 1, ferredoxin encoded by the FD gene 5 has identity of 53.8% to ferredoxin encoded by the FD gene 2, ferredoxin encoded by the FD gene 5 has identity of 62.9% to ferredoxin encoded by the FD gene 3, and ferredoxin encoded by the FD gene 5 has identity of 62.3% to ferredoxin encoded by the FD gene 4.

The FD gene 1 may comprise an amino acid sequence that has 70% or higher identity, preferably 80% or higher identity, more preferably 90% or higher identity, further preferably 95% identity, and most preferably 98% or higher identity to the amino acid sequence as shown in SEQ ID NO: 2, and it may also encode a protein that functions as ferredoxin. The FD gene 2 may comprise an amino acid sequence that has 70% or higher identity, preferably 80% or higher identity, more preferably 90% or higher identity, further preferably 95% identity, and most preferably 98% or higher identity to the amino acid sequence as shown in SEQ ID NO: 4, and it may also encode a protein that functions as ferredoxin. The FD gene 3 may comprise an amino acid sequence that has 70% or higher identity, preferably 80% or higher identity, more preferably 90% or higher identity, further preferably 95% identity, and most preferably 98% or higher identity to the amino acid sequence as shown in SEQ ID NO: 6, and it may also encode a protein that functions as ferredoxin. The FD gene 4 may comprise an amino acid sequence that has 70% or higher identity, preferably 80% or higher identity, more preferably 90% or higher identity, further preferably 95% identity, and most preferably 98% or higher identity to the amino acid sequence as shown in SEQ ID NO: 8, and it may also encode a protein that functions as ferredoxin.

The value of identity can be calculated based on default setting using the BLASTN or BLASTX program equipped with the BLAST algorithm. Specifically, the value of identity is determined by calculating the number of amino acid residues that completely match the others when a pairwise alignment analysis is conducted for a pair of amino acid sequences and then determining the proportion of the number of such residues in all the amino acid residues compared.

The FD gene 1, the FD gene 2, the FD gene 3, and the FD gene 4 are not limited to genes encoding the amino acid sequences as shown in SEQ ID NOs: 2, 4, 6, and 8, respectively. The FD gene 1, the FD gene 2, the FD gene 3, or the FD gene 4 may comprise an amino acid sequence derived from the amino acid sequence as shown in SEQ ID NO: 2, 4, 6, or 8 by deletion, substitution, addition, or insertion of 1 to 10 amino acids, preferably 1 to 8 amino acids, more preferably 1 to 6 amino acids, and further preferably 1 to 3 amino acids, and it may encode a protein that functions as ferredoxin.

Further, the FD gene 1, the FD gene 2, the FD gene 3, and the FD gene 4 are not limited to genes comprising the nucleotide sequences as shown in SEQ ID NOs: 1, 3, 5, and 7, respectively. The FD gene 1, the FD gene 2, the FD gene 3, or the FD gene 4 may hybridize under stringent conditions to all or a part of a complementary strand of DNA comprising the nucleotide sequence shown in SEQ ID NO: 1, 3, 5, or 7, and such gene may encode a protein that functions as ferredoxin. Under “stringent conditions,” a so-called specific hybrid is formed, but a non-specific hybrid is not formed. For example, such conditions can be adequately determined with reference to the Molecular Cloning: A Laboratory Manual (Third Edition). Specifically, stringency can be set based on the temperature and the concentration of salts contained in a solution for southern hybridization, and the temperature and the concentration of salts contained in a solution for a washing step of southern hybridization.

A method for preparing DNA comprising a nucleotide sequence that encodes an amino acid sequence derived from the amino acid sequence as shown in SEQ ID NO: 2, 4, 6, or 8 by deletion, substitution, addition, or insertion of predetermined amino acids or DNA comprising a nucleotide sequence other than the nucleotide sequence as shown in SEQ ID NO: 1, 3, 5, or 7 is not particularly limited, and any conventional technique can adequately be employed. For example, predetermined nucleotides can be substituted via site-directed mutagenesis. Examples of site-directed mutagenesis include T. Kunkel's site-directed mutagenesis (Kunkel, T. A. Proc. Nat. Acad. Sci., U.S.A., 82, 488-492, 1985) and the Gapped duplex method. Moreover, mutagenesis can also be carried out using a mutagenesis kit using site-directed mutagenesis (e.g., Mutan-K (Takara Shuzo Co., Ltd.) and Mutan-G (Takara Shuzo Co., Ltd.)) or a LA PCR in vitro Mutagenesis series kit (Takara Shuzo Co., Ltd.).

Once the nucleotide sequence of a ferredoxin gene is specified, the gene can be isolated in accordance with a conventional technique. For example, a ferredoxin gene may be entirely synthesized based on the specified nucleotide sequence. Alternatively, primers may be designed based on the thus specified nucleotide sequence, and the ferredoxin gene of interest can then be isolated by PCR using the genome of, for example, Nostoc punctiforme PCC 73102, as a template and the primers.

Whether or not a protein comprising an amino acid sequence other than the amino acid sequence as shown in SEQ ID NO: 2, 4, 6, or 8 functions as ferredoxin can be examined in accordance with a conventional technique. When a target protein is purified and found to produce NADPH from NADP⁺, for example, the target protein can be determined as functioning as ferredoxin.

### [Ferredoxin Reductase Gene]

A ferredoxin reductase gene encodes ferredoxin-NADP⁺ reductase (FNR). Ferredoxin reductase genes that can be used in the predetermined combinations described above are 3 types of ferredoxin reductase genes selected from among many ferredoxin reductase genes possessed by blue-green algae. Such 3 types of ferredoxin genes are referred to as the FDR gene 1, the FDR gene 2, and the FDR gene 3.

The FDR gene 1 encodes a protein [c1] or [d1]:

[c1] a protein comprising the amino acid sequence as shown in SEQ ID NO: 10; or

[d1] a protein comprising an amino acid sequence having identity of 60% or higher to the amino acid sequence as shown in SEQ ID NO: 10 and functioning as ferredoxin reductase.

The amino acid sequence as shown in SEQ ID NO: 10 is registered as the amino acid sequence of FAD-dependent pyridine nucleotide-disulfide oxidoreductase derived from Nostoc punctiforme PCC 73102 (Accession Number: YP_001865390 (or WP_012408465)). SEQ ID NO: 9 shows the nucleotide sequence encoding the amino acid sequence as shown in SEQ ID NO: 10.

The FDR gene 2 encodes a protein [c2] or [d2]:

[c2] a protein comprising the amino acid sequence as shown in SEQ ID NO: 12; or

[d2] a protein comprising an amino acid sequence having identity of 60% or higher to the amino acid sequence as shown in SEQ ID NO: 12 and functioning as ferredoxin reductase.

The amino acid sequence as shown in SEQ ID NO: 12 is registered as the amino acid sequence of the oxidoreductase FAD/NAD(P)-binding subunit derived from Nostoc punctiforme PCC 73102 (Accession Number: YP_001866231 (or WP_012409282)). SEQ ID NO: 11 shows the nucleotide sequence encoding the amino acid sequence as shown in SEQ ID NO: 12.

The FDR gene 3 encodes a protein [c3] or [d3]:

[c3] a protein comprising the amino acid sequence as shown in SEQ ID NO: 14; or

[d3] a protein comprising an amino acid sequence having identity of 60% or higher to the amino acid sequence as shown in SEQ ID NO: 14 and functioning as ferredoxin reductase.

The amino acid sequence as shown in SEQ ID NO: 14 is registered as the amino acid sequence of phycocyanobilin:ferredoxin oxidoreductase derived from Nostoc punctiforme PCC 73102 (Accession Number: YP_001868825 (or WP_012411826)). SEQ ID NO: 13 shows the nucleotide sequence encoding the amino acid sequence as shown in SEQ ID NO: 14.

The FDR gene 1 may comprise an amino acid sequence that has 70% or higher identity, preferably 80% or higher identity, more preferably 90% or higher identity, further preferably 95% identity, and most preferably 98% or higher identity to the amino acid sequence as shown in SEQ ID NO: 10, and it may also encode a protein that functions as ferredoxin reductase. The FDR gene 2 may comprise an amino acid sequence that has 70% or higher identity, preferably 80% or higher identity, more preferably 90% or higher identity, further preferably 95% identity, and most preferably 98% or higher identity to the amino acid sequence as shown in SEQ ID NO: 12, and it may also encode a protein that functions as ferredoxin reductase. The FDR gene 3 may comprise an amino acid sequence that has 70% or higher identity, preferably 80% or higher identity, more preferably 90% or higher identity, further preferably 95% identity, and most preferably 98% or higher identity to the amino acid sequence as shown in SEQ ID NO: 14, and it may also encode a protein that functions as ferredoxin reductase.

The value of identity can be calculated based on default setting using the BLASTN or BLASTX program equipped with the BLAST algorithm. Specifically, the value of identity is determined by calculating the number of amino acid residues that completely match the others when a pairwise alignment analysis is conducted for a pair of amino acid sequences and then determining the proportion of the number of such residues in all the amino acid residues compared.

The FDR gene 1, the FDR gene 2, and the FDR gene 3 are not limited to genes encoding the amino acid sequences as shown in SEQ ID NOs: 10, 12, and 14, respectively. The FDR gene 1, the FDR gene 2, or the FDR gene 3 may comprise an amino acid sequence derived from the amino acid sequence as shown in SEQ ID NO: 10, 12, or 14 by deletion, substitution, addition, or insertion of 1 to 50 amino acids, preferably 1 to 40 amino acids, more preferably 1 to 30 amino acids, and further preferably 1 to 20 amino acids, and it may encode a protein that functions as ferredoxin reductase.

Further, the FDR gene 1, the FDR gene 2, and the FDR gene 3 are not limited to genes comprising the nucleotide sequences as shown in SEQ ID NOs: 9, 11, and 13, respectively. The FDR gene 1, the FDR gene 2, or the FDR gene 3 may hybridize under stringent conditions to all or a part of a complementary strand of DNA comprising the nucleotide sequence as shown in SEQ ID NO: 9, 11, or 13, and it may encode a protein that functions as ferredoxin reductase. Under “stringent conditions,” a so-called specific hybrid is formed, but a non-specific hybrid is not formed. For example, such conditions can be adequately determined with reference to the Molecular Cloning: A Laboratory Manual (Third Edition). Specifically, stringency can be set based on the temperature and the concentration of salts contained in a solution for southern hybridization, and the temperature and the concentration of salts contained in a solution for a washing step of southern hybridization.

A method for preparing DNA comprising a nucleotide sequence that encodes an amino acid sequence derived from the amino acid sequence as shown in SEQ ID NO: 10, 12, or 14 by deletion, substitution, addition, or insertion of predetermined amino acids or DNA comprising a nucleotide sequence other than the nucleotide sequence as shown in SEQ ID NO: 9, 11, or 13 is not particularly limited, and any conventional technique can adequately be employed. For example, predetermined nucleotides can be substituted via site-directed mutagenesis. Examples of site-directed mutagenesis include T. Kunkel's site-directed mutagenesis (Kunkel, T. A. Proc. Nat. Acad. Sci., U.S.A., 82, 488-492, 1985) and the Gapped duplex method. Moreover, mutagenesis can also be carried out using a mutagenesis kit using site-directed mutagenesis (e.g., Mutan-K (Takara Shuzo Co., Ltd.) and Mutan-G (Takara Shuzo Co., Ltd.)) or a LA PCR in vitro Mutagenesis series kit (Takara Shuzo Co., Ltd.).

Once the nucleotide sequence of a ferredoxin reductase gene is specified, the gene can be isolated in accordance with a conventional technique. For example, a ferredoxin reductase gene derived from a predetermined organism may be entirely synthesized based on the specified nucleotide sequence. Alternatively, primers may be designed based on the thus specified nucleotide sequence, and the ferredoxin gene of interest can then be isolated by PCR using the genome of a predetermined organism as a template and the primers.

Whether or not a protein comprising an amino acid sequence other than the amino acid sequence as shown in SEQ ID NO: 10, 12, or 14 functions as ferredoxin reductase can be examined in accordance with a conventional technique. When a target protein is purified and found to produce oxidized ferredoxin and NADPH from reduced ferredoxin and NADP⁺, respectively, for example, the target protein can be determined as functioning as ferredoxin reductase.

### [Combinations of Ferredoxin Gene and Ferredoxin Reductase Gene]

Combinations of the ferredoxin genes (the FD genes 1 to 4) and the ferredoxin reductase genes (the FDR genes 1 to 3) are as follows:

the FD gene 1 in combination with the FDR gene 1;

the FD gene 1 in combination with the FDR gene 2;

the FD gene 2 in combination with the FDR gene 1;

the FD gene 2 in combination with the FDR gene 2;

the FD gene 2 in combination with the FDR gene 3;

the FD gene 3 in combination with the FDR gene 2

the FD gene 3 in combination with the FDR gene 3;

the FD gene 4 in combination with the FDR gene 1; or

the FD gene 4 in combination with the FDR gene 2.

A recombinant microorganism comprising the genes in any of the 9 combinations described above acquires alkane productivity superior to that of a microorganism that does not comprise the genes in such combination (e.g., a recombinant microorganism that comprises the genes in a different combination).

Among the 9 different combinations described above, in particular, a recombinant microorganism comprising the FD gene 3 in combination with the FDR gene 2, the FD gene 2 in combination with the FDR gene 3, or the FD gene 4 in combination with the FDR gene 2 exhibits alkane productivity superior to that of a recombinant microorganism comprising the genes in a different combination. In addition, a recombinant microorganism comprising the FD gene 3 in combination with the FDR gene 2 exhibits the best alkane productivity among others.

Among the ferredoxin genes derived from Nostoc punctiforme PCC 73102, the FD gene 5 would not exert the effects of improving alkane productivity in combination with any of the ferredoxin reductase genes (i.e., the FDR genes 1 to 3) described above.

### <Recombinant Microorganism>

A microorganism into which the ferredoxin gene and the ferredoxin reductase gene described above are to be introduced is a microorganism capable of synthesizing alkane or a recombinant microorganism to which the ability to synthesize alkane has been imparted.

Examples of a microorganism capable of synthesizing alkane include Synechococcus elongatus PCC7942, S. elongatus PCC6301, Synechocystis sp. PCC6803, Prochlorococcus marinus CCMP1986, Anabaena variabilis ATCC29413, Nostoc punctiforme PCC73102, Gloeobacter violaceus PCC7421, Nostoc sp. PCC7120, Cyanothece sp. PCC7425, and Cyanothece sp. ATCC51142 (reference: Science 30 Jul. 2010, Vol. 329, No. 5991, pp. 559-562).

An example of a recombinant microorganism to which the ability to synthesize alkane has been imparted is a recombinant microorganism prepared by introducing an alkane synthase gene isolated from the above microorganism capable of synthesizing alkane.

Examples of alkane synthase genes that can be used include the alkS gene isolated from Nostoc sp. ATCC27347 (PCC7120) and the genes described in Science 30 Jul. 2010, Vol. 329, No. 5991, pp. 559-562 and WO 2009/140695. More specific examples of alkane synthase genes that can be used include the alkane synthase genes isolated from Nostoc punctiforme PCC73102, Synechococcus elongates PCC7942, Synechocystis sp. PCC6803, Cyanothece sp. ATCC51142, Acaryochlloris marina MBIC11017, Gleobacter violaceus PCC7421, and Prochlorococcus marinus str. MIT9303.

As described in Science 30 Jul. 2010, Vol. 329, No. 5991, pp. 559-562, the decarbonylase gene and the acyl-ACP reductase gene may be within the scope of the “alkane synthase gene.”

Examples of decarbonylase genes include 4 types of genes of: [1] decarbonylase represented by Npun_R1711 of Nostoc punctiforme (Science above); [2] decarbonylase related to aldehyde dehydrogenase (JP Patent No. 5,867,586); [3] long-chain alkane synthase represented by the Cer1 gene of Arabidopsis thaliana (Plant Cell, 24, 3106-3118, 2012); and [4] P450 alkane synthase represented by the CYP4G1 gene of Drosophila (PNAS, 109, 37, 14858-14863, 2012).

Specific examples of [1] include Npun_R0380 of Nostoc punctiforme (the Npun_R1711 paralog), Nos7524_4304 of Nostoc sp., Anacy_3389 of Anabaena cylindrica, Aazo_3371 of Anabaena azollae, Cylst_0697 of Cylindrospermum stagnale, Glo7428_0150 of Gloeocapsa sp., Ca17507_5586 of Calothrix sp., FIS3754_06310 of Fischerella sp., Mic7113_4535 of Microcoleus sp., Chro_1554 of Chroococcidiopsis thermalis, GEI7407_1564 of Geitlerinema sp., and Cyan8802_0468 of Cyanothece sp.

Specific examples of [2] include: BAE77705, BAA35791, BAA14869, BAA14992, BAA15032, BAA16524, BAE77705, BAA15538, and BAA15073 derived from the Escherichia coli K-12 W3110 strain; YP_001268218, YP_001265586, YP_001267408, YP_001267629, YP_001266090, YP_001270490, YP_001268439, YP_001267367, YP_001267724, YP_001269548, YP_001268395, YP_001265936, YP_001270470, YP_001266779, and YP_001270298 derived from the Pseudomonas putida_F1 strain; NP_388129, NP_389813, NP_390984, NP_388203, NP_388616, NP_391658, NP_391762, NP_391865, and NP_391675 derived from the Bacillus subtilis 168 strain; NP_599351, NP_599725, NP_601988, NP_599302, NP_601867, and NP_601908 derived from the Corynebacterium glutamicum ATCC13032 strain; YP_001270647 derived from the Lactobacillus reuteri DSM20016 strain; NP_010996, NP_011904, NP_015264, NP_013828, NP_009560, NP_015019, NP_013893, NP_013892, and NP_011902 derived from Saccharomyces cerevisiae; XP_002548035, XP_002545751, XP_002547036, XP_002547030, XP_002550712, XP_002547024, XP_002550173, XP_002546610, and XP_002550289 derived from the Candida tropicalis MYA-3404 strain; XP_460395, XP_457244, XP_457404, XP_457750, XP_461954, XP_462433, XP_461708, and XP_462528 derived from the Debaryomyces hansenii CBS767 strain; XP_002489360, XP_002493450, XP_002491418, XP_002493229, XP_002490175, XP_002491360, and XP_002491779 derived from the Pichia pastoris GS 115 strain; NP_593172, NP_593499, and NP_594582 derived from Schizosaccharomyces pombe; XP_001822148, XP_001821214, XP_001826612, XP_001817160, XP_001817372, XP_001727192, XP_001826641, XP_001827501, XP_001825957, XP_001822309, XP_001727308, XP_001818713, XP_001819060, XP_001823047, XP_001817717, and XP_001821011 derived from the Aspergillus oryzae RIB40 strain; NP_001150417, NP_001105047, NP_001147173, NP_001169123, NP_001105781, NP_001157807, NP_001157804, NP_001105891, NP_001105046, NP_001105576, NP_001105589, NP_001168661, NP_001149126, and NP_001148092 derived from Zea mays; NP_564204, NP_001185399, NP_178062, NP_001189589, NP_566749, NP_190383, NP_187321, NP_190400, NP_001077676, and NP_175812 derived from Arabidopsis thaliana; NP_733183, NP_609285, NP_001014665, NP_649099, NP_001189159, NP_610285, and NP_610107 derived from Drosophila melanogaster; NP_001006999, XP_001067816, XP_001068348, XP_001068253, NP_113919, XP_001062926, NP_071609, NP_071852, NP_058968, NP_001011975, NP_115792, NP_001178017, NP_001178707, NP_446348, NP_071992, XP_001059375, XP_001061872, and NP_001128170 derived from Rattus norvegicus; NP_036322, NP_001193826, NP_001029345, NP_000684, NP_000680, NP_000683, NP_000681, NP_001071, NP_000687, NP_001180409, NP_001173, NP_000682, NP_000373, NP_001154976, NP_000685, and NP_000686 derived from Homo sapiens; and KPN_02991, KPN_1455, and KPN_4772 derived from the Klebsiella sp. NBRC100048 strain.

Specific examples of [3] include AT1G02190 and AT1G02205 (CER1) of Arabidopsis thaliana, 4330012 of Oryza sativa, 101252060 of Solanum lycopersicum, CARUB_v10008547 mg of Capsella rubella, 106437024 of Brassica napus, 103843834 of Brassica rapa, EUTSA_v10009534 mg of Eutrema salsugineum, 104810724 of Tarenaya hassleriana, 105773703 of Gossypium raimondii, TCM_042351 of Theobroma cacao, 100243849 of Vitis vinifera, 105167221 of Sesamum indicum, 104442848 of Eucalyptus grandis, 103929751 of Pyrus bretschneideri, 107618742 of Arachis ipaensis, and 103428452 of Malus domestica.

Specific examples of [4] include CYP4G1 of Drosophila melanogaster, 101887882 of Musca domestica, AaeL_AAEL006824 of Aedes aegypti, and AgaP_AGAP000877 of Anopheles gambiae.

An acyl-ACP reductase gene is not particularly limited, and a gene encoding acyl-ACP reductase registered as EC 1.2.1.80 can be used. Examples of acyl-ACP reductase genes include Synpcc7942_1594 of Synechococcus elongatus, M744_09025 of Synechococcus sp., LEP3755_23580 of Leptolyngbya sp., Glo7428_0151 of Gloeocapsa sp., Nos7107_1027 of Nostoc sp., Ava_2534 of Anabaena variabilis, IJ00_07395 of Calothrix sp., Cri9333_4415 of Crinalium epipsammum, and FIS3754_06320 of Fischerella sp.

Examples of alkane synthase genes that can be used include the decarbonylase gene derived from the N. punctiforme PCC 73102 strain and the acyl-ACP reductase gene derived from the Synechococcus elongatus PCC 7942 strain.

The decarbonylase gene derived from the N. punctiforme PCC 73102 strain encodes a protein comprising the amino acid sequence as shown in SEQ ID NO: 16. A decarbonylase gene may encode a protein comprising an amino acid sequence having identity of 60% or higher, preferably 70% or higher, more preferably 80% or higher, further preferably 90% or higher, still further preferably 95% or higher, and most preferably 98% or higher to the amino acid sequence as shown in SEQ ID NO: 16 and having decarbonylase activity.

The acyl-ACP reductase gene derived from the Synechococcus elongatus PCC 7942 strain encodes a protein comprising the amino acid sequence as shown in SEQ ID NO: 18. An acyl-ACP reductase gene may encode a protein comprising an amino acid sequence having identity of 60% or higher, preferably 70% or higher, more preferably 80% or higher, further preferably 90% or higher, still further preferably 95% or higher, and most preferably 98% or higher to the amino acid sequence as shown in SEQ ID NO: 18 and having acyl ACP reductase activity.

The value of identity can be calculated based on default setting using the BLASTN or BLASTX program equipped with the BLAST algorithm. Specifically, the value of identity is determined by calculating the number of amino acid residues that completely match the others when a pairwise alignment analysis is conducted for a pair of amino acid sequences and then determining the proportion of the number of such residues in all the amino acid residues compared.

The decarbonylase gene and the acyl-ACP reductase gene are not limited to genes encoding the amino acid sequences as shown in SEQ ID NOs: 16 and 18, respectively. The decarbonylase gene or the acyl-ACP reductase gene may encode a protein that comprises an amino acid sequence derived from the amino acid sequence as shown in SEQ ID NO: 16 or 18 by deletion, substitution, addition, or insertion of 1 to 50 amino acids, preferably 1 to 40 amino acids, more preferably 1 to 30 amino acids, and further preferably 1 to 20 amino acids, and it may encode a protein that functions as decarbonylase or acyl-ACP reductase.

Further, the decarbonylase gene and the acyl-ACP reductase gene are not limited to genes comprising the nucleotide sequences as shown in SEQ ID NOs: 15 and 17, respectively. The decarbonylase gene or the acyl-ACP reductase gene may hybridize under stringent conditions to all or a part of a complementary strand of DNA that comprises the nucleotide sequence as shown in SEQ ID NO: 15 or 17, and it may encode a protein that functions as decarbonylase or acyl-ACP reductase. Under “stringent conditions,” a so-called specific hybrid is formed, but a non-specific hybrid is not formed. For example, such conditions can be adequately determined with reference to the Molecular Cloning: A Laboratory Manual (Third Edition). Specifically, stringency can be set based on the temperature and the concentration of salts contained in a solution for southern hybridization, and the temperature and the concentration of salts contained in a solution for a washing step of southern hybridization.

A method for preparing DNA comprising a nucleotide sequence that encodes an amino acid sequence derived from the amino acid sequence as shown in SEQ ID NO: 16 or 18 by deletion, substitution, addition, or insertion of predetermined amino acids or DNA comprising a nucleotide sequence other than the nucleotide sequence as shown in SEQ ID NO: 15 or 17 is not particularly limited, and any conventional technique can adequately be employed. For example, predetermined nucleotides can be substituted via site-directed mutagenesis. Examples of site-directed mutagenesis include T. Kunkel's site-directed mutagenesis (Kunkel, T. A. Proc. Nat. Acad. Sci., U.S.A., 82, 488-492, 1985) and the Gapped duplex method. Moreover, mutagenesis can also be carried out using a mutagenesis kit using site-directed mutagenesis (e.g., Mutan-K (Takara Shuzo Co., Ltd.) and Mutan-G (Takara Shuzo Co., Ltd.)) or a LA PCR in vitro Mutagenesis series kit (Takara Shuzo Co., Ltd.).

An alkane synthase gene is not limited to the acyl-ACP reductase gene described above. A gene encoding an enzyme that synthesizes aldehyde serving as a substrate of the decarbonylase described above can be used.

Examples of genes that can be used include genes encoding long-chain fatty acid acyl-CoA reductase (EC.1.2.1.50), such as plu2079 (luxC) of Photorhabdus luminescens, PAU_02514 (luxC) of Photorhabdus asymbiotica, VF_A0923 (luxC) of Aliivibrio fischeri, VIBHAR_06244 of Vibrio campbellii, and Swoo_3633 of Shewanella woodyi. For example, genes encoding acyl-CoA reductase described in JP 2015-226477 A, such as 100776505 and 100801815 of Glycine max, can also be used. In addition to the genes described above, any gene encoding an enzyme that can synthesize aldehyde can also be used without limitation. Examples of genes that can be used include genes encoding enzymes such as alcohol dehydrogenase (EC.1.1.1.1), alcohol oxidase (EC. 1.1.3.13), aldehyde dehydrogenase (EC. 1.2.1.3), and carboxylate reductase (EC. 1.2.99.6).

Microorganisms into which the alkane synthase gene is to be introduced are not particularly limited. Examples thereof include Escherichia coli and Klebsiella bacteria. As a microorganism into which the alkane synthase gene is to be introduced, Corynebacterium glutamicum disclosed in Appl. Environ. Microbiol., 79 (21): 6776-6783, 2013 (November) can be used. This literature discloses a recombinant Corynebacterium glutamicum that has acquired the ability to produce fatty acid. In addition, Mortierella alpina disclosed in Food Bioprocess Technol., 2011, 4: 232-240 can be used as a microorganism into which the alkane synthase gene is to be introduced. Mortierella alpina is used for arachidonic acid fermentation at the industrial level. In the literature mentioned above, such microorganism is subjected to metabolic engineering. Further, Yarrowia lipolytica disclosed in Trends in Biotechnology, Vol. 34, No. 10, pp. 798-809 can be used as a microorganism into which the alkane synthase gene is to be introduced.

In addition, bacteria of Lipomyces, Pseudozyma, Rhodosporidium, Rhodococcus and the like can be used as microorganisms into which the alkane synthase gene is to be introduced. While a method for introducing the alkane synthase gene into such microorganisms is not particularly limited, genetic recombination techniques involving the use of genome editing systems, such as CRISPR/Cas or TALEN, can be adopted.

When the alkane synthase gene is to be introduced into an yeast, further, an yeast species is not particularly limited. Examples include yeast of Pichia, such as Pichia stipites, yeast of Saccharomyces, such as Saccharomyces cerevisiae, and yeast of Candida, such as Candida tropicalis and Candida prapsilosis.

The ferredoxin gene and the ferredoxin reductase gene described above may be introduced into the genome of host microorganisms capable of synthesizing alkane. Thus, recombinant microorganisms that can be used can be prepared. The ferredoxin gene and the ferredoxin reductase gene may be simultaneously introduced into a host. Alternatively, either thereof may be first introduced, and the other may then be introduced. When the alkane synthase gene described above is to be introduced into a host microorganism that is not capable of synthesizing alkane, in addition to the ferredoxin gene and the ferredoxin reductase gene described above, the alkane synthase gene may be introduced into the host simultaneously with the ferredoxin gene and the ferredoxin reductase gene. The alkane synthase may be introduced before or after the ferredoxin gene and the ferredoxin reductase gene are introduced.

When the ferredoxin gene and the ferredoxin reductase gene are introduced into a host, for example, a DNA fragment containing a ferredoxin gene and a ferredoxin reductase gene is ligated to an expression vector and preferably a multicopy vector, which functions in a host microorganism, so as to prepare recombinant DNA, and the recombinant DNA is then introduced into the microorganism for transformation. Examples of an expression vector that can be used herein include, but are not particularly limited to, a plasmid vector, and a chromosome transfer vector that can be incorporated into the genome of a host organism. An expression vector to be used herein is not particularly limited, and it may be adequately selected from all available expression vectors depending on host microorganisms. In addition, examples of an expression vector include plasmid DNA, bacteriophage DNA, retrotransposon DNA, and artificial chromosome DNA (YAC: yeast artificial chromosome).

Examples of plasmid DNA include YCp-type Escherichia coli-yeast shuttle vectors such as pRS413, pRS414, pRS415, pRS416, YCp50, pAUR112, and pAUR123, YEp-type Escherichia coli-yeast shuttle vectors such as pYES2 and YEp13, YIp-type Escherichia coli-yeast shuttle vectors such as pRS403, pRS404, pRS405, pRS406, pAUR101, and pAUR135, Escherichia coli-derived plasmids (ColE-based plasmids such as pBR322, pBR325, pUC18, pUC19, pUC118, pUC119, pTV118N, pTV119N, pBluescript, pHSG298, pHSG396, and pTrc99A, p15A-based plasmids such as pACYC177 and pACYC184, and pSC101-based plasmids such as pMW118, pMW119, pMW218 and pMW219), Agrobacterium-derived plasmids (e.g., pBI101), and Bacillus subtilis-derived plasmids (e.g., pUB110 and pTP5). Examples of phage DNA include λ phage (Charon4A, Charon21A, EMBL3, EMBL4, λgt10, λgt11, and λZAP), ϕλ174, M13mp18, and M13mp19. An example of retrotransposon is a Ty factor. An example of a YAC vector is pYACC2. An animal virus vector, such as a retrovirus or a vaccinia virus vector, and an insect virus vector, such as a baculovirus vector, can also be used.

It is necessary that a ferredoxin gene and a ferredoxin reductase gene be incorporated into an expression vector, so that each gene can be expressed. To this end, the ferredoxin gene and the ferredoxin reductase gene are ligated to predetermined promoters and then incorporated into a vector, so that the genes are expressed under the control of the predetermined promoters in a host organism into which the ferredoxin gene and the ferredoxin reductase gene are to be introduced. In addition to the ferredoxin gene and the ferredoxin reductase gene, a promoter, a terminator, a cis element such as an enhancer, according to need, a splicing signal, a poly-A addition signal, a selection marker, a ribosomal binding sequence (SD sequence), and the like can be ligated to an expression vector. In addition, examples of selection markers include antibiotic-resistant genes, such as an ampicillin-resistant gene, a kanamycin-resistant gene, and a hygromycin-resistant gene.

As a transformation method involving the use of an expression vector, a conventional technique can adequately be employed. Examples of transformation techniques include a calcium chloride method, a competent cell method, a protoplast or spheroplast method, and an electrical pulse method.

The ferredoxin gene and the ferredoxin reductase gene can be introduced, so as to increase the number of copies thereof. Specifically, the ferredoxin gene and the ferredoxin reductase gene may be introduced, so that the multiple copies thereof are present on the chromosomal DNA of a microorganism. The multiple copies of the ferredoxin gene and the ferredoxin reductase gene can be introduced into the chromosomal DNA of a microorganism by homologous recombination using a sequence comprising multiple copies of such genes present on the chromosomal DNA as a target.

The expression levels of the ferredoxin gene and the ferredoxin reductase gene can be enhanced by various methods, such as substitution of expression regulatory sequences (i.e. promoters) of the endogenous or introduced ferredoxin gene and ferredoxin reductase gene with those capable of increasing the expression levels of the genes or introduction of a regulator sequence that increases the expression level of a predetermined gene. Examples of such a promoter that enables high-level gene expression include, but are not particularly limited to, a lac promoter, a trp promoter, a trc promoter, and a pL promoter. Furthermore, the endogenous or introduced ferredoxin gene and ferredoxin reductase gene can be altered, so that the genes can be expressed at higher levels as a result of introduction of mutations into expression control regions for the genes.

### <Alkane Production>

As described above, alkane can be synthesized with good productivity with the use of recombinant microorganisms resulting from introduction of the predetermined ferredoxin gene and the predetermined ferredoxin reductase gene into microorganisms capable of synthesizing alkane or recombinant microorganisms that have acquired the ability to synthesize alkane.

With the use of a system involving the use of a microorganism capable of synthesizing alkane or a recombinant microorganism to which the ability to synthesize alkane has been imparted, alkane can be produced in a medium suitable for these microorganisms via culture in such medium. More specifically, the ability of alkane synthase to synthesize alkane can be improved, and as a result, alkane productivity can be improved.

When the ability of alkane synthase to synthesize alkane is to be improved, the ability of alkane synthase to convert aldehyde into alkane is to be improved. Specifically, the efficiency of an alkane synthesis reaction induced by alkane synthase is improved because of the presence of ferredoxin and ferredoxin reductase in a predetermined combination.

A target alkane to be produced herein is not particularly limited. Examples thereof include alkane having 9 to 20, preferably 14 to 17, and more preferably 13 to 16 carbon atoms. These alkane examples are highly viscous liquids, which can be used for light oil (diesel fuel) or aviation fuel. Such alkane can be isolated from the above reaction system in which the recombinant microorganism had been cultured in accordance with a general method, and it can then be purified.

The method described in Engineering in Life Sciences, Vol. 16, page 1, 53-59 “Biosynthesis of chain-specific alkanes by metabolic engineering in Escherichia coli” may be adopted, so that an alkane having a short chain length can be synthesized.

### EXAMPLES

Hereafter, the present invention is described in greater detail with reference to the examples, although the technical scope of the present invention is not limited to these examples.

### Example 1

### [1. Objectives]

In Example 1, a ferredoxin gene and a ferredoxin reductase gene in the N. puntioforme PCC 73102 strain, the origin of which is the same as that of the decarbonylase gene to be introduced into a host to provide the capacity for alkane synthesis, were deduced, these genes were co-introduced (co-expressed) into E. coli in a predetermined combination, and the influence imposed on alkane production was then inspected. Genes exhibiting 70% or higher amino acid sequence homology to that of alr0784, all1430, asr2513, all2919, and all4148 annotated with reference to ferredoxin, all4121 annotated with reference to ferredoxin reductase, and alr3707 annotated with reference to ferredoxin oxidoreductase of the Nostoc sp. PCC 7120 strain whose genome had been disclosed since the early stage were selected as the genes to be inspected.

When ferredoxin and ferredoxin reductase of the Nostoc sp. PCC 7120 strain are searched for with the use of KEGG, 10 types of ferredoxin reductases and 8 types of ferredoxins are identified as candidates, and 12 types of ferredoxin reductases and 24 types of ferredoxins are identified as candidates for Nostoc punctioforme. That is, it is not possible to narrow the range of the ferredoxin gene and the ferredoxin reductase gene derived from N. puntioforme on the basis of the annotation information stored in databases.

In Example 1, accordingly, ferredoxin and ferredoxin reductase candidates of the Nostoc sp. PCC 7120 strain were deduced on the basis of the amino acid sequences of the ferredoxin and the ferredoxin reductase derived from spinach used for the decarbonylase enzyme test described in Science 30 Jul. 2010, Vol. 329, No. 5991, pp. 559-562. On the basis of the ferredoxin and the ferredoxin reductase deduced for the Nostoc sp. PCC 7120 strain, the range of ferredoxin and ferredoxin reductase derived from N. puntioforme was narrowed down.

The ferredoxin derived from spinach is registered under Accession Number: M35660, and the ferredoxin reductases derived from spinach are registered under Accession Numbers: M86349, M86349, and X64351.

### [2. Materials and Method]

### 2.1 Reagents

Reagents that are not identified with the manufactures were purchased from Nacalai tesque.

### 2.2 Strains

The E. coli Rosetta (DE3) strain was purchased from Novagen, and the E. coli BL21 (DE3) strain and the E. coli JM109 strain were purchased from Takara Bio Inc. The Klebesiella sp. NBRC100048 strain was provided by the National Institute of Technology and Evaluation.

### 2.3 Preparation of M9YE Medium

To a mixture of 10 ml of a 10% solution of Bacto Yeast extract (Difco), 50 ml of a 20% glucose solution, 1 ml of 1 M MgSO₄, 1 ml of a 1% thiamine solution, 0.1 ml of a 1 M CaCl₂ solution, and 100 ml of 10×M9 medium (manufactured by MP biomedicals), a necessary antibiotic solution was added, and the volume of the mixture was adjusted to 1,000 ml with the addition of sterile water.

### 2.4 Synthesis of Artificial Gene

From the genome sequence of the N. punctiforme PCC 73102 strain, YP_001865390, YP_001866231, YP_001868825, YP_001864061, YP_001864105, YP_001864826, YP_001865513, and YP_001867060 were selected as the genes exhibiting 70% or higher amino acid sequence homology to alr0784 (annotation: ferredoxin), all1430 (annotation: heterocyst ferredoxin, fdxH), asr2513 (annotation: ferredoxin, fdxB), all2919 (annotation: ferredoxin), all4148 (annotation: ferredoxin I, petF), all4121 (annotation: ferredoxin-NADP(+) reductase, petH), and alr3707 (annotation: phycocyanobilin, ferredoxin oxidoreductase) of the Nostoc sp. PCC 7120 strain, and synthesis thereof was consigned to GeneScript. At the time of synthesis, codons were optimized based on the codon usage frequency of S. cerevisiae or E. coli, and the codons were designated as shown in Table 1 separately from the IDs of the original genome sequences.

TABLE 1

| | | | Artificial gene (synthetic DNA with optimized codon) | | |
|---|---|---|---|---|---|
| | | Nostoc puntioforme | | | Codon |
| Nostoc sp. PCC 7120 | | PCC 73102 | | Gene | optimization |
| Annotation | ID | ID | DNA | abbreviation | target |
| Ferredoxin | asr2513 | YP_001864061 | 4.Fd_NP_YP_001864061 | Fd4 | S. cerevisiae |
| | all1430 | YP_001864105 | 5.Fd_NP_YP_001864105 | Fd5 | S. cerevisiae |
| | all2919 | YP_001864826 | 6.Fd_NP_YP_001864826 | Fd6 | S. cerevisiae |
| | alr0784 | YP_001865513 | 7.Fd_NP_YP_001865513 | Fd7 | S. cerevisiae |
| | | | 7.Fd_NP_YP_001865513b | | E. coli |
| | all4148 | YP_001867060 | 8.Fd_NP_YP_001867060 | Fd8 | S. cerevisiae |
| Ferredoxin | all4121 | YP_001866231 | 2.FDR_NP_YP_001866231 | FDR2 | S. cerevisiae |
| reductase | | | 2.FDR_NP_YP_001866231b | | E. coli |
| Ferredoxin | alr3707 | YP_001865390 | 1.FDR_NP_YP_001865390 | FDR1 | S. cerevisiae |
| oxidoreductase | | YP_001868825 | 3.FDR_NP_YP_001868825 | FDR3 | S. cerevisiae |

Concerning the abbreviations shown in Table 1, “Fd4” represents the FD gene 5, “Fd5” represents the FD gene 1, “Fd6” represents the FD gene 2, “Fd7” represents the FD gene 3, “Fd8” represents the FD gene 4, “FDR1” represents the FDR gene 1, “FDR2” represents the FDR gene 2, and “FDR3” represents the FDR gene 3. The nucleotide sequences of the coding regions of the FD genes 1 to 5 and the FDR genes 1 to 3 and the amino acid sequences encoded by the nucleotide sequences are summarized in Table 2.

TABLE 2

| Gene | Nucleotide sequence | Amino acid sequence |
|---|---|---|
| FD gene 1 (Fd5) | SEQ ID NO: 1 | SEQ ID NO: 2 |
| FD gene 2 (Fd6) | SEQ ID NO: 3 | SEQ ID NO: 4 |
| FD gene 3 (Fd7) | SEQ ID NO: 5 | SEQ ID NO: 6 |
| FD gene 4 (Fd8) | SEQ ID NO: 7 | SEQ ID NO: 8 |
| FD gene 5 (Fd4) | SEQ ID NO: 19 | SEQ ID NO: 20 |
| FDR gene 1 (FDR1) | SEQ ID NO: 9 | SEQ ID NO: 10 |
| FDR gene 2 (FDR2) | SEQ ID NO: 11 | SEQ ID NO: 12 |
| FDR gene 3 (FDR3) | SEQ ID NO: 13 | SEQ ID NO: 14 |

It should be noted that these sequences are the sequences stored in databases, which are different from the sequences resulting from codon optimization in this example.

The synthesized DNA was introduced into the pUC57 vector and provided by GeneScript in that state. The XbaI site and the BamHI site are added to the both ends of 2.FDR_NP_YP_001866231b and 7.Fd_NP_YP_001865513b whose codons were optimized for E. coli.

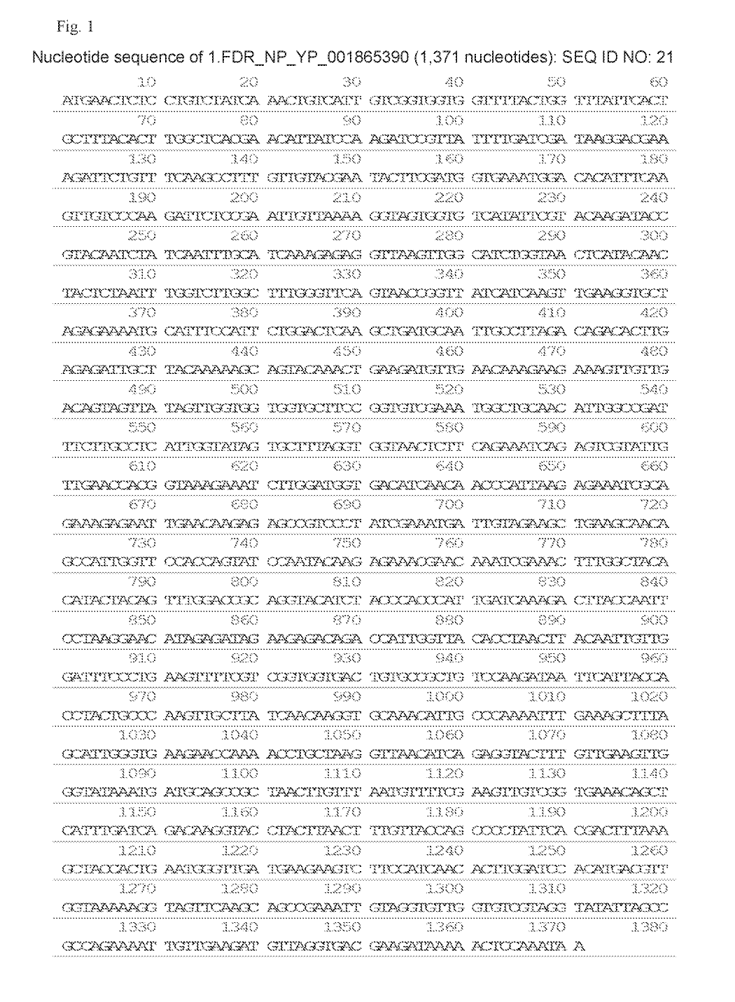

FIGS. 1 to 10 show the nucleotide sequences of artificial genes designed and synthesized in this example (SEQ ID NOs: 21 to 30) as shown in Table 3.

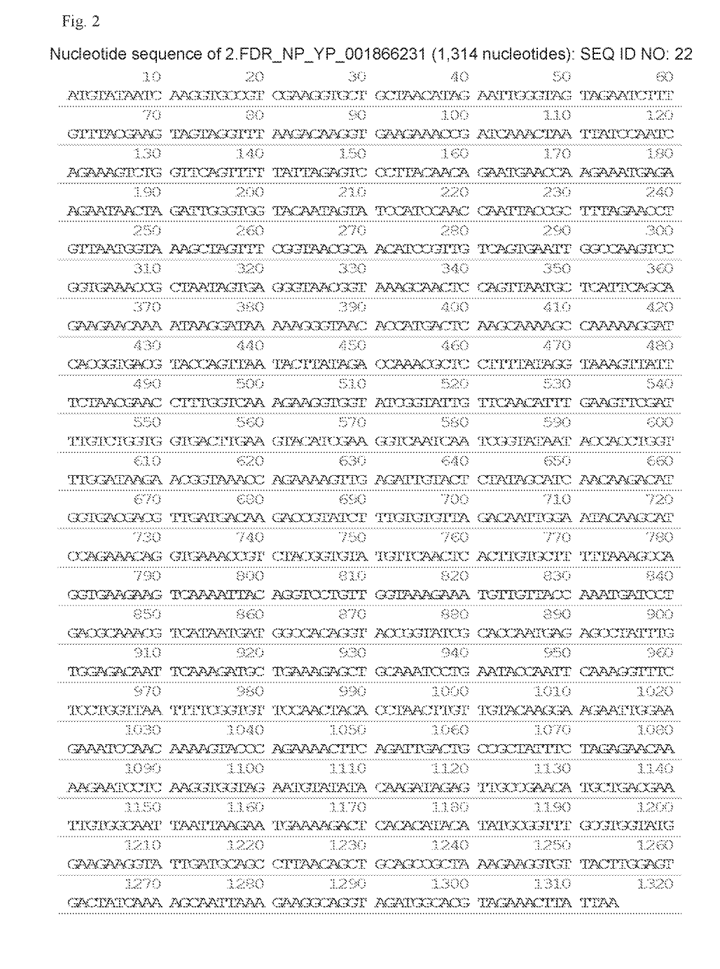

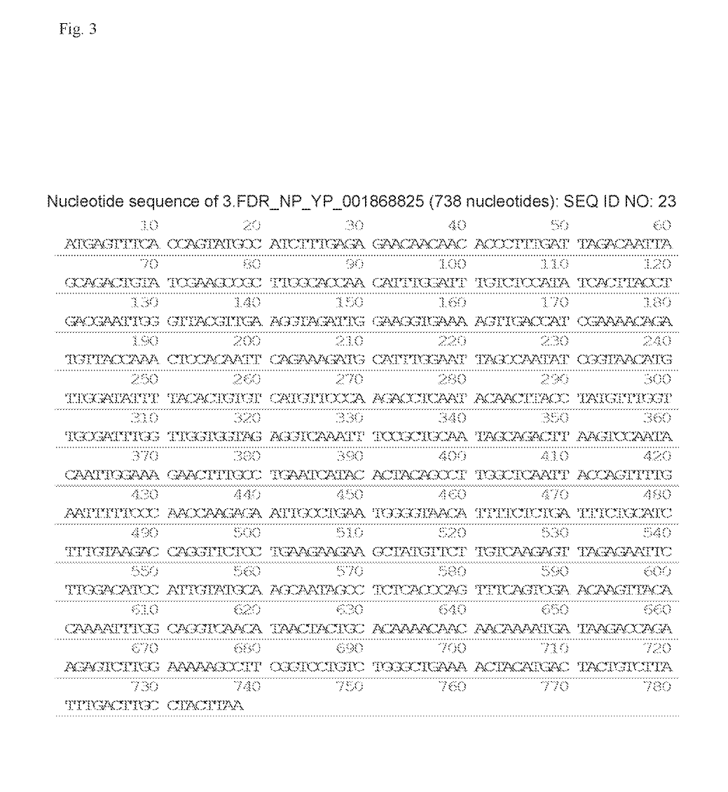

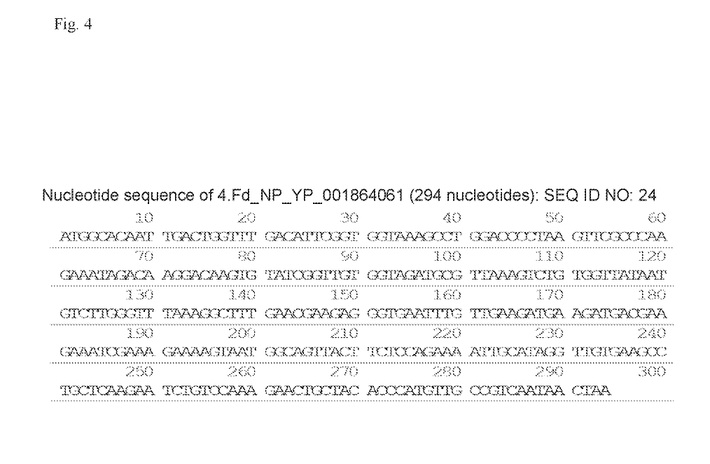

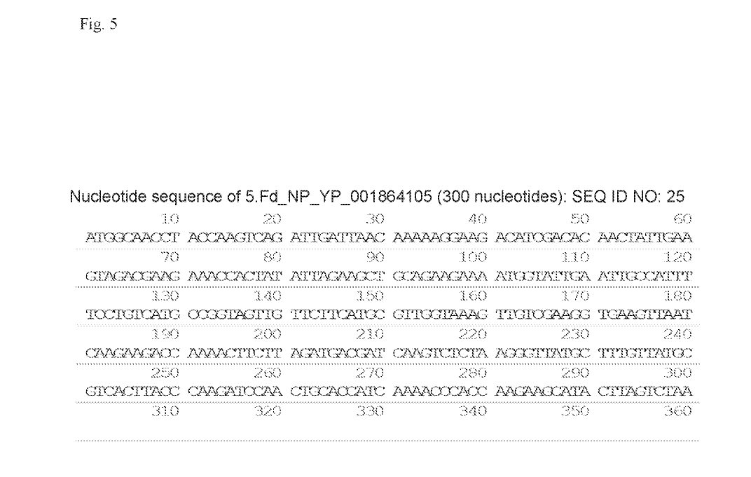

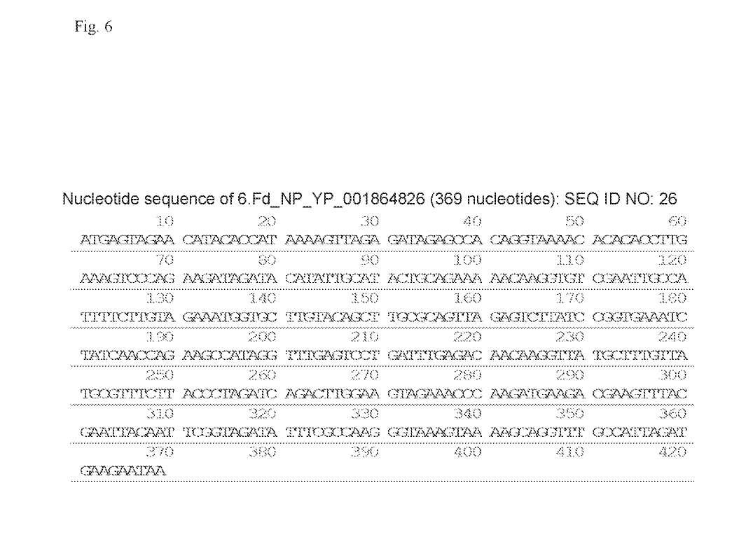

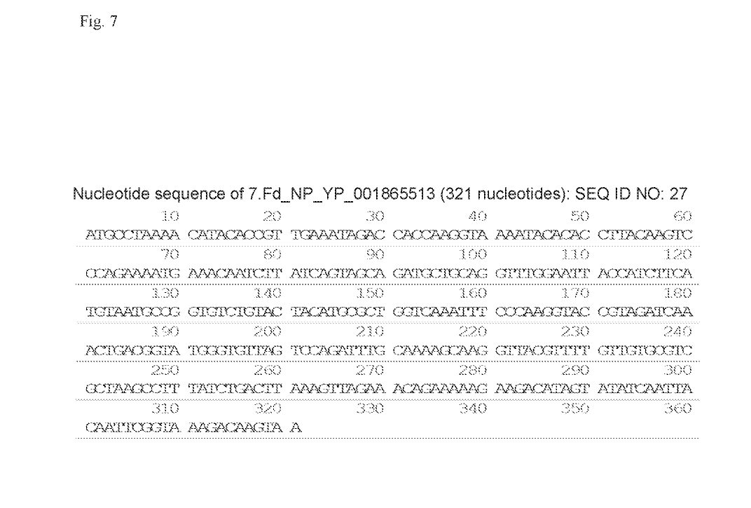

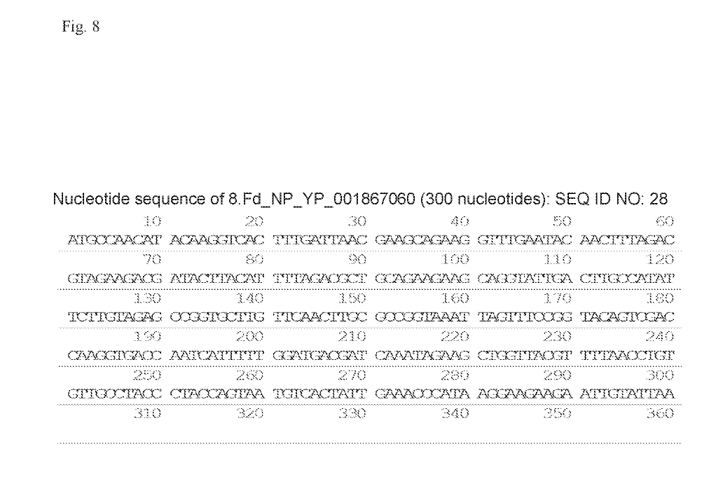

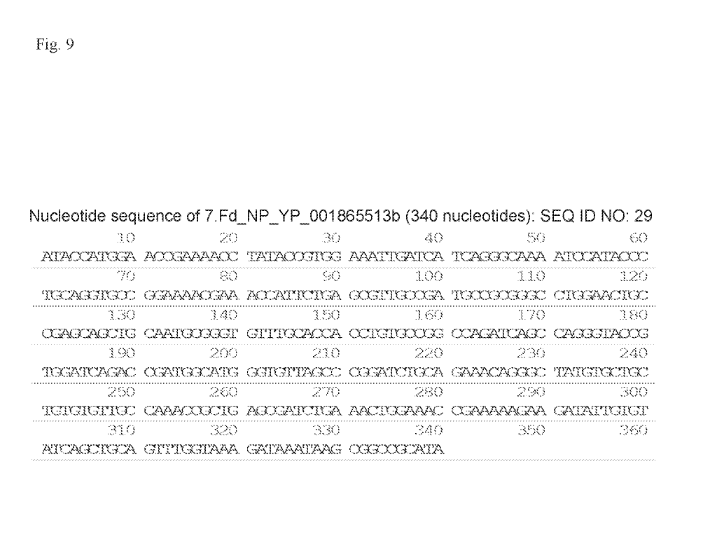

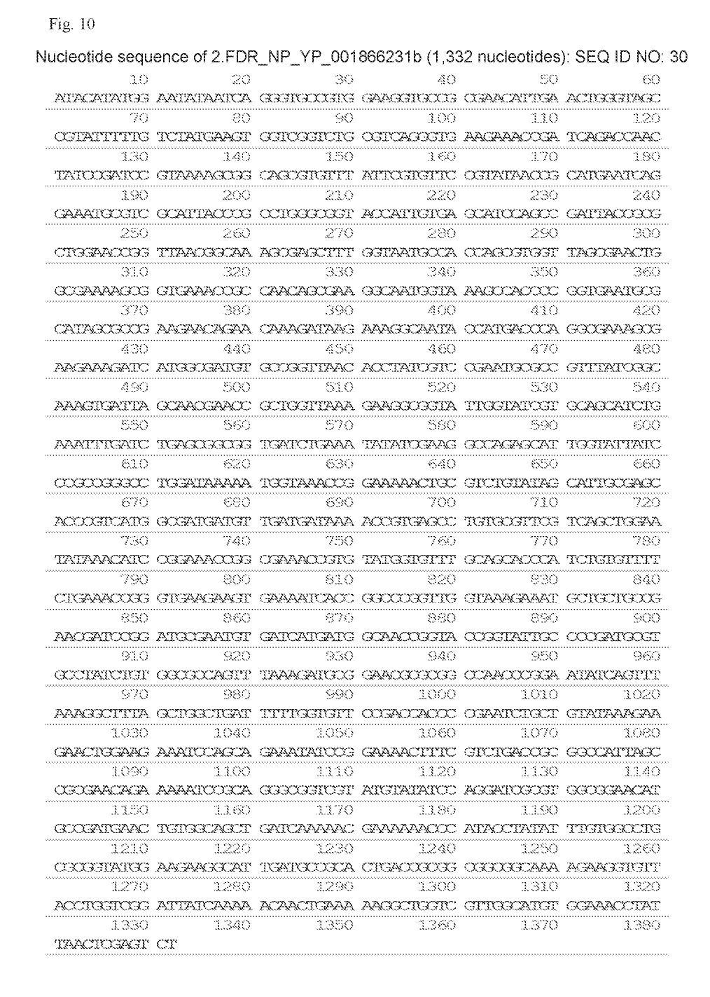

TABLE 3

| Artificial gene | FIG. No. |
|---|---|
| 1.FDR_NP_YP_001865390 | FIG. 1 |
| 2.FDR_NP_YP_001866231 | FIG. 2 |
| 3.FDR_NP_YP_001868825 | FIG. 3 |
| 4.Fd_NP_YP_001864061 | FIG. 4 |
| 5.Fd_NP_YP_001864105 | FIG. 5 |
| 6.Fd_NP_YP_001864826 | FIG. 6 |
| 7.Fd_NP_YP_001865513 | FIG. 7 |
| 8.Fd_NP_YP_001867060 | FIG. 8 |
| 7.Fd_NP_YP_001865513b | FIG. 9 |
| 2.FDR_NP_YP_001866231b | FIG. 10 |

### 2.5 Preparation of Plasmid for Gene Introduction

2.5.1 Preparation of pCDF-FDRs

PCR was carried out using template DNAs and primers in the combination as shown in Table 4 (the nucleotide sequences of the primers are described below, and the same applies hereinbelow), the amplified DNA fragments were inserted into pCDFDuet-1 (Novagen) digested with the NdeI and PacI restriction enzymes with the use of the In-Fusion HD Cloning kit (Invitrogen), and the resulting plasmids were designated as pCDF-FDR1, pCDF-FDR2, and pCDF-FDR3. pCDFDuet-1 has a streptomycin-resistant gene. When culturing the transformed pCDF-FDRs, accordingly, selection was performed with the addition of 50 mg/l streptomycin.

TABLE 4

| Prepared | Combination of PCR components | | |
|---|---|---|---|
| plasmid | Template DNA | Primer 1 | Primer 2 |
| pCDF-FDR1 | 1.FDR_NP_YP_001865390 | FDR1-inf-F | FDR1-inf-R |
| pCDF-FDR2 | 2.FDR_NP_YP_001866231 | FDR2-inf-F | FDR2-inf-R |
| pCDF-FDR3 | 3.FDR_NP_YP_001868825 | FDR3-inf-F | FDR3-inf-R |

PCR conditions were 92° C. for 2 minutes, a cycle of 95° C. for 20 seconds, 55° C. for 20 seconds, and 72° C. for 1 minute repeated 25 times, and 72° C. for 3 minutes, followed by 16° C. The composition of the PCR reaction solution is shown in Table 5.

TABLE 5

| | |
|---|---|
| Template(1 ng/ml) | 1 μl |
| 10 × Pfu Ultra ll reaction buffer | 5 μl |
| dNTP mix. (25 mM each) | 0.5 μl |
| Primer 1(10 mM) | 1 μl |
| Primer 2(10 mM) | 1 μl |
| Pfu Ultra II fusion HS DNA polymerase | 1 μl |
| Sterile water | 40.5 μl |
| Total | 50 μl |

2.5.2 Preparation of pCDF-FDR-Fds

With the use of synthetic DNA comprising a ferredoxin gene and a ferredoxin reductase gene synthesized by GeneScript (mounted on the pUC57 vector) as a template, PCR was carried out using the primers in the combination shown in Table 6.

TABLE 6

| | | | | Vector side | | |
|---|---|---|---|---|---|---|
| | | | | pCDF-FDR1 | pCDF-FDR2 | pCDF-FDR3 |
| | Template | Primer | Gene | FDR1 | FDR2 | FDR3 |
| Passenger side | 4.Fd_NP_YP_001864061 | Fd4-inf-F | Fd4 | pCDFDuet-FDR1-Fd4 | pCDFDuet-FDR2-Fd4 | pCDFDuet-FDR3-Fd4 |
| (ferredoxin) | | Fd4-inf-R | | | | |
| | 5.Fd_NP_YP_001864105 | Fd5-inf-F | Fd5 | pCDFDuet-FDR1-Fd5 | pCDFDuet-FDR2-Fd5 | pCDFDuet-FDR3-Fd5 |
| | | Fd5-inf-R | | | | |
| | 6.Fd_NP_YP_001864826 | Fd6-inf-F | Fd6 | pCDFDuet-FDR1-Fd6 | pCDFDuet-FDR2-Fd6 | pCDFDuet-FDR3-Fd6 |
| | | Fd6-inf-R | | | | |
| | 7.Fd_NP_YP_001865513 | Fd7-inf-F | Fd7 | pCDFDuet-FDR1-Fd7 | pCDFDuet-FDR2-Fd7 | pCDFDuet-FDR3-Fd7 |
| | | Fd7-inf-R | | | | |
| | 8.Fd_NP_YP_001867060 | Fd8-inf-F | Fd8 | pCDFDuet-FDR1-Fd8 | pCDFDuet-FDR2-Fd8 | pCDFDuet-FDR3-Fd8 |
| | | Fd8-inf-R | | | | |

After the PCR-amplified fragment was separated via agarose gel electrophoresis, the separated fragment was purified using the MinElute PCR purification kit (QIAGEN) to prepare DNAs of the Fd4, Fd5, Fd6, Fd7, and Fd8 genes.

Subsequently, pCDF-FDR1, pCDF-FDR2, and pCDF-FDR3 prepared in 2.5.1 were digested with the NcoI and NotI restriction enzymes, and Fd4, Fd5, Fd6, Fd7, and Fd8 were inserted with the use of the In-Fusion HD Cloning kit (Invitrogen). Thus, pCDF-FDR-Fds expressing both the ferredoxin gene and the ferredoxin reductase gene shown in Table 6 were prepared.

2.5.3 Preparation of pCDF-lacP-Fd7-FDR2

As described below, the use of FDR2 in combination with Fd7 was the most effective for improving alkane productivity in E. coli. Accordingly, a plasmid was constructed, so as to enable evaluation of such combination in Klebliella.

The pTV118N plasmid (Takara Bio Inc.) was digested with the NcoI restriction enzyme and subjected to 1.5% agarose gel electrophoresis. A 3.2-kb DNA fragment was then cleaved. Subsequently, PCR was carried out using the template and the primers in the combination shown in Table 7, the amplified 0.3-kb and 1.3-kb DNA fragments were purified, and the purified fragments were allowed to bind to a 3.2-kb fragment with the use of the In-Fusion HD Cloning kit (Invitrogen). The resulting plasmid was designated as pTV-Fd-FDR.

TABLE 7

| Amplified | | Combination of PCR components | | |
|---|---|---|---|---|
| gene | DNA length | Template | Primer 1 | Primer 2 |
| Fd7 | 0.3 kb | 7.Fd_NP_YP_001865513b | FDR-Fw | FDR-Rw |
| FDR2 | 1.3 kb | 2.FDR_NP_YP_001866231b | FDR2-Fw | FDR2-Rw |

The resulting pTV-Fd-FDR was digested with ApaLI (Takara Bio Inc.) and a DNA fragment of about 2.8 kb was separated via electrophoresis. The pCDFDuet-1 plasmid (Novagen) was treated with restriction enzymes DrdI (New England Biolabs Inc.) and XbaI (Takara Bio Inc.), and a DNA fragment of about 1.7 kb was separated via electrophoresis. The 2.8-kb and 1.7-kb fragments were blunt-ended with the use of the Blunting kit (Takara Bio Inc.), and these fragments were ligated to each other with the use of the Ligation convenience kit (NipponGene). The resulting plasmid was designated as pCDF-lacP-Fd7-FDR2.

2.5.4 pRSF-NpAD-SeAR

pRSF-NpAD-SeAR comprising the decarbonylase gene derived from the N. punctiforme PCC 73102 strain and the acyl-ACP reductase gene derived from the Synechococcus elongatus PCC7942 strain mounted on pRSF-1b (Novagen) was prepared. pRSF-NpAD-SeAR was prepared in the manner described in Experiment Example 1 below.

2.5.5 pTV-NpAD-SeAR-amp and pTV-NpAD-SeAR-kan

pTV-NpAD-SeAR-amp and pTV-NpAD-SeAR-kan comprising the decarbonylase gene derived from the N. punctiforme PCC 73102 strain and the acyl-ACP reductase gene derived from the Synechococcus elongatus PCC7942 strain mounted on pTV118N (Takara Bio Inc.) were prepared. pTV-NpAD-SeAR-amp and pTV-NpAD-SeAR-kan were prepared in the manner described in Experiment Example 1 below.

### 2.6 Preparation of Transformant

Transformants were prepared with the use of plasmids in the combination shown in Table 8. The pCDF-FDR-Fd prepared in this example has been optimized in accordance with the codon usage frequency of the yeast. In order to suppress the influence caused by the difference from the codon usage frequency of E. coli, accordingly, the E. coli Rosetta (DE3) strain was used as a host for the pCDF Duet-FDR-Fd. In contrast, the Klebsiella sp. 100048 strain does not possess T7 RNA polymerase. Accordingly, the lac promoter was used, and the E. coli JM109 strain was used for comparison. Rosetta (DE3) competent cells (Novagen) or ECOS competent E. coli JM109 cells (NipponGene) were transformed in accordance with the instructions attached to the kit. The Klebesiella sp. 100048 strain was transformed in the manner described in Experiment Example 1 below.

TABLE 8

| Strain | | Plasmid | Plasmid | Antibiotics |
|---|---|---|---|---|
| No. | Host | introduced 1 | introduced 2 | added |
| 1 | E. coli Rosetta (DE3) | — | — | — |
| 2 | E. coli Rosetta (DE3) | pRSF-NpAD-SeAR | — | Amp |
| 3 | E. coli Rosetta (DE3) | pRSF-NpAD-SeAR | pCDFDuet-FDR1-Fd4 | Amp, Sm |
| 4 | E. coli Rosetta (DE3) | pRSF-NpAD-SeAR | pCDFDuet-FDR1-Fd5 | Amp, Sm |
| 5 | E. coli Rosetta (DE3) | pRSF-NpAD-SeAR | pCDFDuet-FDR1-Fd6 | Amp, Sm |
| 6 | E. coli Rosetta (DE3) | pRSF-NpAD-SeAR | pCDFDuet-FDR1-Fd7 | Amp, Sm |
| 7 | E. coli Rosetta (DE3) | pRSF-NpAD-SeAR | pCDFDuet-FDR1-Fd8 | Amp, Sm |
| 8 | E. coli Rosetta (DE3) | pRSF-NpAD-SeAR | pCDFDuet-FDR2-Fd4 | Amp, Sm |
| 9 | E. coli Rosetta (DE3) | pRSF-NpAD-SeAR | pCDFDuet-FDR2-Fd5 | Amp, Sm |
| 10 | E. coli Rosetta (DE3) | pRSF-NpAD-SeAR | pCDFDuet-FDR2-Fd6 | Amp, Sm |
| 11 | E. coli Rosetta (DE3) | pRSF-NpAD-SeAR | pCDFDuet-FDR2-Fd7 | Amp, Sm |
| 12 | E. coli Rosetta (DE3) | pRSF-NpAD-SeAR | pCDFDuet-FDR2-Fd8 | Amp, Sm |
| 13 | E. coli Rosetta (DE3) | pRSF-NpAD-SeAR | pCDFDuet-FDR3-Fd4 | Amp, Sm |
| 14 | E. coli Rosetta (DE3) | pRSF-NpAD-SeAR | pCDFDuet-FDR3-Fd5 | Amp, Sm |
| 15 | E. coli Rosetta (DE3) | pRSF-NpAD-SeAR | pCDFDuet-FDR3-Fd6 | Amp, Sm |
| 16 | E. coli Rosetta (DE3) | pRSF-NpAD-SeAR | pCDFDuet-FDR3-Fd7 | Amp, Sm |
| 17 | E. coli Rosetta (DE3) | pRSF-NpAD-SeAR | pCDFDuet-FDR3-Fd8 | Amp, Sm |
| 18 | E. coli JM109 | — | — | — |
| 19 | E. coli JM109 | pTV-NpAD-SeAR-amp | — | Amp |
| 20 | E. coli JM109 | pTV-NpAD-SeAR-amp | pCDF-lacP-Fd7-FDR2 | Amp, Sm |
| 21 | Klebsiella sp. 100048 | — | — | — |
| 22 | Klebsiella sp. 100048 | pTV-NpAD-SeAR-kan | — | Kan |
| 23 | Klebsiella sp. 100048 | pTV-NpAD-SeAR-kan | pCDF-lacP-Fd7-FDR2 | Kan, Sm |

### 2.7 Culture and Analysis of Product

Transformants were inoculated into a 14-ml round tube (BD Falcon) containing 3 ml of an LB Broth Miller medium (Luria-Bertani, Difco) containing necessary antibiotics, and shake culture was performed using a three-stage culture vessel (MW-312, ABLE) at 100 strokes/min for 18 hours at 37° C. The preculture solution was inoculated into 3 ml of antibiotic-containing M9YE at 1%, and culture was conducted in a disposable glass test tube (f16 mm×150 mm, IWAKI) using the same culture vessel at 30° C. and 90 strokes/min for 2 to 3 days. In this culture, IPTG was added to a final concentration of 1 mM 4 hours after inoculation.

To the culture solution, the same amount (3 ml) of ethyl acetate was added 2 or 3 days after the initiation of culture, and they were mixed with the vortex mixture for 10 seconds. After the mixture was centrifuged with the use of the LC-230 centrifuge (TOMY) at room temperature for 10 minutes at 2,000 rpm, 1 ml of the ethyl acetate layer was transferred to a GC/MS vial, 10 ml of the internal standard solution (1 l/ml R-(−)-2-octanol/ethanol) was added, and the culture vessel was tightly closed. GC/MS quantification was carried out under the conditions described in Experimental Example 1 below.

### [3. Results]

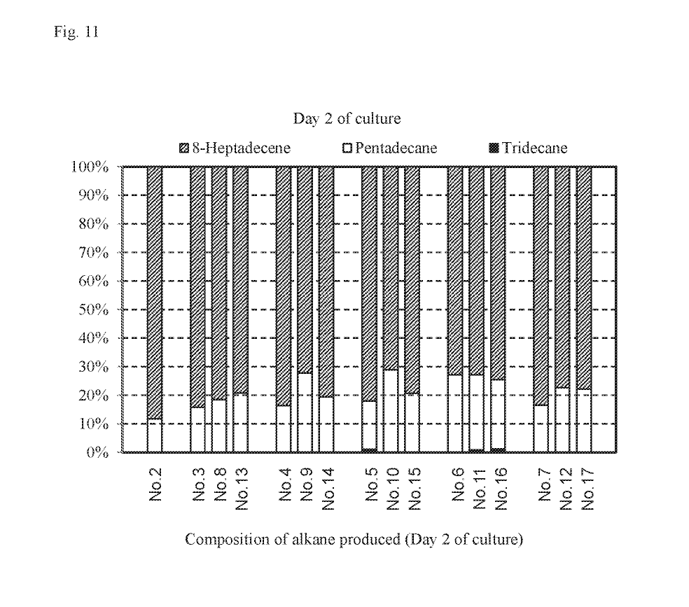

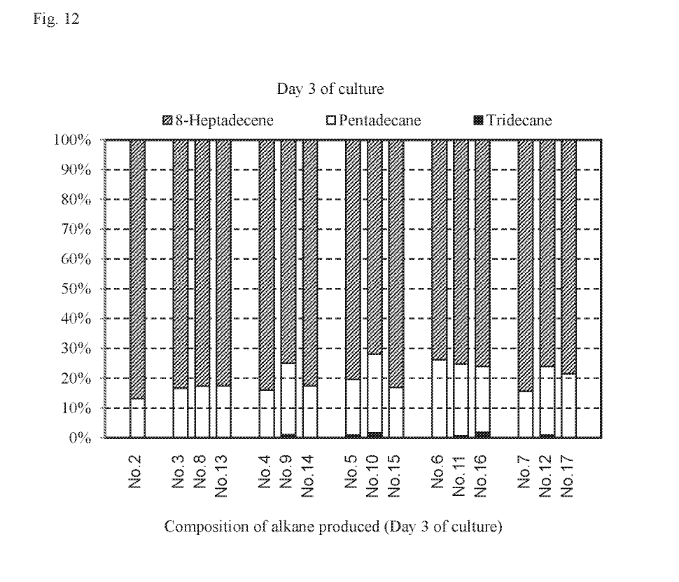

Strain Nos. 2 to 17 were cultured (n=2 or 3), the amount of alkane production was measured via GC/MS, and the average thereof is shown in FIGS. 11 and 12. As shown in FIGS. 11 and 12, production of 8-heptadecane, pentadecane, and a very small amount of tridecane was observed. While the amounts of 8-heptadecane and of pentadecane produced were not consistent, a major product was 8-heptadecane derived from oleic acid. While the amounts of 8-heptadecane and pentadecane produced were consistent 2 days and 3 days after the initiation of culture irrespective of strains, the amount of pentadecane produced was likely to increase upon introduction of a ferredoxin gene and a ferredoxin reductase gene.

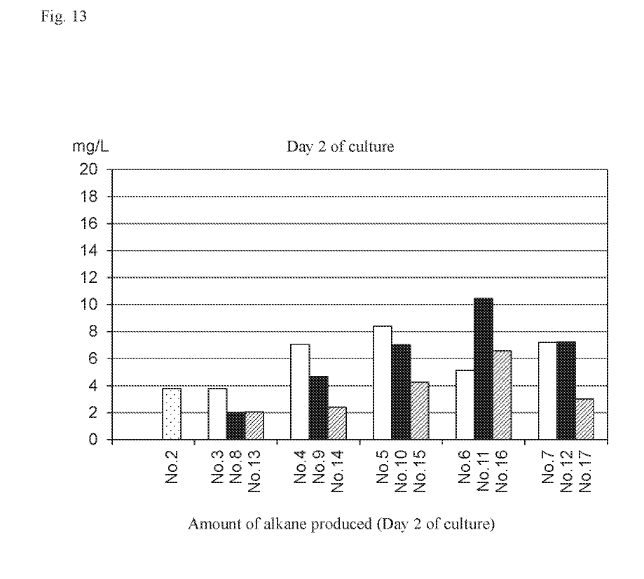

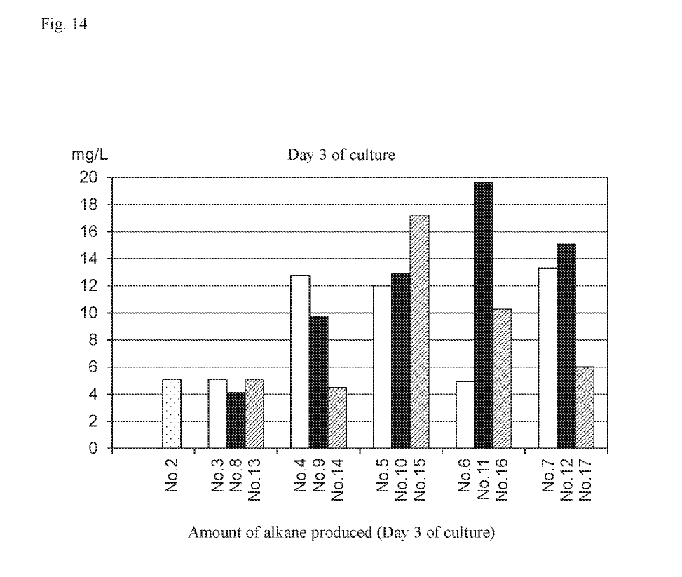

The total amounts of 3 types of alkanes produced by the E. coli Rosetta strains (Strain Nos. 2 to 17) were compared. The results of comparison are shown in FIGS. 13 and 14. Also, the amount of alkane production 3 days after the initiation of culture was compared with the amount of Strain No. 2. The summary of comparison is shown in Table 9.

TABLE 9

| | | | Ferredoxin (oxi)reductase | | |
|---|---|---|---|---|---|
| | | | YP_001865390 | YP_001866231 | YP_001868825 |
| | | | FDR1 | FDR2 | FDR3 |
| ferredoxin | YP_001864061 | Fd4 | x | x | x |
| | YP_001864105 | Fd5 | 2.5 times | 1.9 times | x |
| | YP_001864 826 | Fd6 | 2.4 times | 2.5 times | 3.4 times |
| | YP_001865513 | Fd7 | x | 3.8 times | 2.0 times |
| | YP_001867060 | Fd8 | 2.6 times | 3.0 times | x |

As shown in FIGS. 13 and 14 and Table 9, the amount of alkane production was found to increase when a ferredoxin gene other than Fd4 (i.e., any of Fd5 to Fd8) was used in combination with a particular type of ferredoxin reductase gene. In other words, FDR2 (YP_001866231), which is a ferredoxin oxidoreductase homolog, was found to interact with any ferredoxin other than Fd4 and produce an increased amount of alkane. In contrast, two types of ferredoxin reductase homologs (i.e., FDR1 (YP_001865390) and FDR3 (YP_001868825)) were found to have ferredoxin selectivity.

In particular, alkane productivity of Fd7 in combination with FDR2, that of Fd6 in combination with FDR3, and that of Fd8 in combination with FDR2 were superior to that of other combinations. Further, the highest alkane productivity was achieved with the use of Fd7 in combination with FDR2.

When Fd6 was used in combination with FDR1 to FDR3, as is apparent from FIGS. 13 and 14, the amount of alkane production 2 days after the initiation of culture was in a descending order from FDR1 to FDR3, but an opposite trend was observed 3 days after the initiation of culture (i.e., the amount of alkane production was in a descending order from FDR3 to FDR1). Regarding other Fd, the trend observed 2 days after the initiation of culture was the same as that observed 3 days after the initiation of culture.

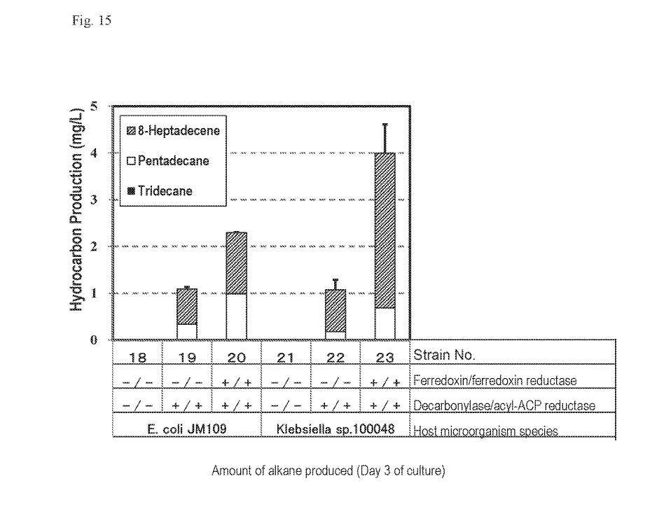

The combination of FDR2 and Fd7 exhibiting the greatest total amount of alkane production among the combinations shown in FIG. 14 and Table 9 was introduced into the Klebsiella sp. 100048 strain, and the amount of alkane production was examined. The results are shown in FIG. 15. While the effects of improving the alkane productivity attained by FDR2 in combination with Fd7 were two times greater in the E. coli JM109 strain, such effects were found to be 4 times greater than the effects in the Klebsiella sp. 100048 strain.

### [Primers]

The nucleotide sequences of the primers used in this example are summarized in Table 10.

TABLE 10

| Primer | Nucleotide sequence | SEQ ID NO: |
|---|---|---|
| FDR1-inf-F | AAGGAGATATACATATGAACTCTCCTGTCTATCAAA | 31 |
| FDR1-inf-R | CAGCAGCCTAGGTTATTATTTGGAGTTTTTATCTTCGTC | 32 |
| FDR2-inf-F | AAGGAGATATACATATGTATAATCAAGGTGCCGTCG | 33 |
| FDR2-inf-R | CAGCAGCCTAGGTTATTAATAAGTTTCTACGTGCCATC | 34 |
| FDR3-inf-F | AAGGAGATATACATATGAGTTTCACCAGTATGCC | 35 |
| FDR3-inf-R | CAGCAGCCTAGGTTATTAAGTAGGCAAGTCAAATAAG | 36 |
| Fd4-inf-F | AGGAGATATACCATGGCACAATTGACTGGTTTG | 37 |
| Fd4-inf-R | AAGCATTATGCGGCCTTAGTTATTGACGGCAACATG | 38 |
| Fd5-inf-F | AGGAGATATACCATGGCAACCTACCAAGTCAG | 39 |
| Fd5-inf-R | AAGCATTATGCGGCCTTAGACTAAGTATGCTTCTTG | 40 |
| Fd6-inf-F | AGGAGATATACCATGAGTAGAACATACACCATA | 41 |
| Fd6-inf-R | AAGCATTATGCGGCCTTATTCTTCATCTAATGGCAAAC | 42 |
| Fd7-inf-F | AGGAGATATACCATGCCTAAAACATACACCGTTG | 43 |
| Fd7-inf-R | AAGCATTATGCGGCCTTACTTGTCTTTACCGAATTG | 44 |
| Fd8-inf-F | AGGAGATATACCATGCCAACATACAAGGTCAC | 45 |
| Fd8-inf-R | AAGCATTATGCGGCCTTAATACAATTCTTCTTCCTTATG | 46 |
| Fd7-Fw | AGGAAACAGACCATGGAACCGAAAACCTATACCGTG | 47 |
| Fd7-Rv | GAAATTGTTATCCGCGGCCGCTTATTTATCTTTACCAAAC | 48 |
| FDR2-Fw | CGGATAACAATTTCACACAGGAAACAGACATGGAATATAATCAGGGTGCCG | 49 |
| FDR2-Rv | GTAATCATGGCCATGTTAATAGGTTTCCACATGCCAACG | 50 |

### Experimental Example 1

Method for Preparing pRSF-NpAD-SeAR

At the outset, the acyl-ACP reductase gene derived from the Synechococcus elongatus PCC 7942 strain (YP_400611) and the decarbonylase gene derived from the Nostoc punctiforme PCC 73102 strain (YP_001865325) were chemically synthesized. These synthetic genes were inserted into the pUC57 EcoRV site and designated as pUC57-SeAAR and pUC57-NpAD, respectively.

Subsequently, PCR was carried out under the conditions described below with the use of pUC57-NpAD and pUC57-SeAAR as templates and Pfu Ultra II Fusion HS DNA Polymerase (STRATAGENE), and the amplified NpADvo and SeAAvo fragments were obtained.

TABLE 11

| Reaction composition: | |
|---|---|
| pUC57-NpAD (30 ng/μl) | 1 μl |
| 10 × Pfu Ultra II reaction buffer | 5 μl |
| dNTP mix. (25 mM each) | 1 μl |
| Primer pRSF-NpAS-inf-F (10 μM) | 2 μl |
| Primer pRSF-NpAS-inf-R (10 μM) | 2 μl |
| Pfu Ultra II fusion HS DNA polymerase | 1 μl |
| Sterile deionized water | 38 μl |
| Total | 50 μl |

TABLE 12

| Reaction composition: | |
|---|---|
| pUC57-SeAAR (1 ng/μl) | 1 μl |
| 10 × Pfu Ultra II reaction buffer | 5 μl |
| dNTP mix (25 mM each) | 1 μl |
| Primer pRSF-SeAR-inf-F (10 μM) | 2 μl |
| Primer pRSF-SeAR-inf-R (10 μM) | 2 μl |
| Pfu Ultra II fusion HS DNA polymerase | 1 μl |
| Sterile deionized water | 38 μl |
| Total | 50 μl |

PCR conditions were 92° C. for 2 minutes, a cycle of 92° C. for 10 seconds, 55° C. for 20 seconds, and 68° C. for 5 minutes repeated 25 times, and 72° C. for 3 minutes, followed by 16° C. The primer sequences are as shown below.

pRSF-NpAS-inf-F primer:
(SEQ ID NO: 51)
5′-cgagctcggcgcgcctgcagATGCAGCAGCTTACAGACCA-3′
pRSF-NpAS-inf-R primer:
(SEQ ID NO: 52)
5′-gcaagcttgtcgacctgcagTTAAGCACCTATGAGTCCGT-3′
pRSF-SeAR-inf-F primer:
(SEQ ID NO: 53)
5′-aaggagatatacatatgATGTTCGGTCTTATCGGTCA-3′
pRSF-SeAR-inf-R primer:
(SEQ ID NO: 54)
5′-ttgagatctgccatatgTCAAATTGCCAATGCCAAGG-3′

Subsequently, PstI-treated pRSF-1b (Novagen) was ligated to the NpADvo fragment using the In-Fusion HD Cloning kit (Invitrogen), the resulting plasmid was digested with NdeI, and the resultant was then connected to the SeAAvo fragment using the same kit.

The vector thus obtained was designated as pRSF-NpAD-SeAR.

Method for preparing pTV-NpAD-SeAR-amp and pTV-NpAD-SeAR-kan

At the outset, PCR was carried out under the conditions described below with the use of pRSF-NpAD-SeAR prepared above as a template and Pfu Ultra II Fusion HS DNA Polymerase (Agilent). The NpAD Fw primer and the NpAD Rv primer were used as the forward primer and the reverse primer to amplify the NpADvo2 fragment. The SeAR Fw primer and the SeAR Rv primer were used as the forward primer and the reverse primer to amplify the SeAAvo2 fragment.

TABLE 13

| Reaction composition: | |
|---|---|
| pRSF-NpAD-SeAR (1 ng/μl) | 1 μl |
| 10 × Pfu Ultra II reaction buffer | 5 μl |
| dNTP mix (25 mM each) | 0.5 μl |
| Forward primer (10 μM) | 1 μl |
| Reverse primer (10 μM) | 1 μl |
| Pfu Ultra II fusion HS DNA polymerase | 1 μl |
| Sterile deionized water | 40.5 μl |
| Total | 50 μl |

PCR conditions were 92° C. for 2 minutes, a cycle of 95° C. for 20 seconds, 60° C. for 20 seconds, and 72° C. for 30 seconds repeated 25 times, and 72° C. for 3 minutes, followed by 16° C. The primer sequences are as shown below.

NpAD Fw primer:
(SEQ ID NO: 55)
5′-AGGAAACAGACGTACATGCAGCAGCTTACAGACCAATC-3′
NpAD Rv primer:
(SEQ ID NO: 56)
5′-GAAATTGTTATCCGCTTAAGCACCTATGAGTCCGTAG-3′
SeAR Fw primer:
(SEQ ID NO: 57)
5′-GCGGATAACAATTTCACACAGGAAACAGACATGTTCGGTCTTATCGG
TCATC-3′
SeAR Rv primer:
(SEQ ID NO: 58)
5′-GTAATCATGGCGTACTCAAATTGCCAATGCCAAGG-3′

Subsequently, NcoI-treated pTV118N (Takara Bio Inc.), the NpADvo2 fragment, and the SeAAvo2 fragment were mixed at a ratio of 1:2:2 by mole, and they were ligated to each other using the In-Fusion HD Cloning kit. The vector thus obtained was designated as pTV-NpAD-SeAR-amp.

The resulting pTV-NpAD-SeAR-amp vector was treated with DraI to obtain a 4,211-bp fragment. Separately, the pRSFDuet vector (Novagen) was treated with BspHI (NEB) to obtain a 877-bp fragment, and the resulting fragment was blunt-ended. The 4,211-bp fragment was ligated to the blunt-ended 877-bp fragment. The resulting vector was designated as pTV-NpAD-SeAR-kan.

Method for Transformation of Klebesiella sp. 100048 Strain

The Klebesiella sp. 100048 strain was inoculated into a 14-ml round tube (BD Falcon) containing 3 ml of a medium, and culture was conducted at 37° C. for 18 hours to prepare a preculture solution. The medium was composed of 10 g of polypeptone (Wako Pure Chemicals), 2 g of an yeast extract (Difco), and 1 g of MgSO₄.7H₂O. The medium composition was dissolved in 0.8 l of deionized water, a pH level of the solution was adjusted to 7.0 with the aid of 1 N hydrochloric acid or a 1 N sodium hydroxide solution, the volume of the solution was adjusted to 1 liter with the addition of deionized water, and the resultant was treated in an autoclave at 121° C. for 15 minutes.

Subsequently, the preculture solution was inoculated into a 100-ml baffled triangular flask containing 30 ml of the same medium in an amount of 1% therein, and culture was conducted at 160 rpm and 37° C. in a two-step culture vessel (type IMF-II-S, Oriental Giken Inc.). Immediately after O.D. 600 of the culture solution reached 0.5 to 0.7, the flask was ice-cooled for 20 minutes, the culture solution was transferred to a 50-ml centrifuge tube, and the culture solution was then centrifuged at 4° C. and 4,000 g for 5 minutes to obtain a cell pellet. The cells were suspended in a 10% glycerol 1 solution that had been ice-cooled in advance. The cell suspension was centrifuged at 4° C. and 4,000 g for 5 minutes, the supernatant was discarded, and the remnant was resuspended in 15 ml of an ice-cooled 10% glycerol solution. This procedure was repeated 3 times to thoroughly wash the cells. In the end, the cells were suspended in 1 ml of an ice-cooled 10% glycerol solution, and the total amount of the suspension was then transferred to an Eppendorf tube. After the cell suspension was centrifuged at 4° C. and 4,000 g for 5 minutes and the supernatant was removed, the remnant was resuspended in 0.12 ml of an ice-cooled 10% glycerol solution, and the suspension was fractionated to fractions of 40 μl each on ice to obtain competent cells. The thus-prepared competent cells were stored at −80° C. before use.

The plasmid solution prepared above (1 μm) was mixed with the competent cells thawed on ice, the resultant was allowed to stand on ice for 1 minute, the resultant was introduced into an ice-cooled electroporation cuvette (GenePulser Cuvette, 0.2 cm), and electroporation was then carried out using GenePulserXcell (BIORAD) at 2.5 kV pulse. SOC medium (1 ml) was added to the cuvette so as to mildly suspend the cells therein, the cell suspension was transferred to a 14-ml round tube (BD Falcon), and shake culture was carried out at the temperature shown in Table 1 for 1 hour. After the completion of culture, the culture solution was applied to various agarose plates containing antibiotics.

Quantification via GC/MS

The recombinants grown on the agarose plate were inoculated into a 14-ml round tube (BD Falcon) containing 3 ml of the medium, and culture was conducted using a three-step culture vessel (MW-312, ABLE) at 130 strokes/min for 18 hours at a predetermined temperature. The thus-prepared preculture solution was inoculated into a disposable glass test tube ((p 16×150 mm, IWAKI) containing 3 ml of the M9YE medium containing antibiotics in an amount of 1% therein, and culture was conducted in the same manner at 90 strokes/min for 4 hours. Thereafter, IPTG (final concentration: 1 mM) was added, and culture was further conducted for 3 days.

After the completion of culture, 1.5 ml of the culture solution was fractionated into an Eppendorf tube, and centrifugation was then conducted with the use of a small-size centrifuge (MX-301, TOMY SEIKO CO., LTD.) at 24° C. and 5,800 g for 1 minute. The supernatant was removed while leaving 50 μl of the supernatant left behind, and the cells were then suspended. Subsequently, 150 μl of ethyl acetate was added, the mixture was vigorously mixed with the use of an Eppendorf multi-sample vortex mixer 5432 for 5 minutes, centrifugation was carried out in the same manner at 24° C. and 13,000 g for 1 minute, and 100 μl of an ethyl acetate layer was then transferred to a GC/MS vial. Thereafter, 50 μl of the internal standard solution (i.e., 0.4% (v/v) of 2-octanol dissolved in 2-propanol) was added, and the resultant was then subjected to GC/MS analysis (7890GC/5975MSD, Agilent). Conditions of analysis are described below.

Of the chromatogram, 71 squares were selected, and the peak areas of tridecane (retention time: 4.376 minutes) and of 2-propanol (retention time: 3.378 minutes) were determined. A calibration curve was prepared based on the ratio of the peak area of tridecane relative to the area of 2-propanol, and the tridecane concentration was determined.

TABLE 14
<Conditions of GC-MS analysis>

Detector: MS
MS zone temperature
MS quad: 150° C.
MS source: 230° C.
Interface temperature: 260° C.
Column: Agilent HP-5MS (0.25 mm ϕ × 30 m;
film thickness: 0.25 μm)
Column temperature: retained at 60° C. for 1 min, raised at 50° C./min,
and retained at 300° C. for 1 min
Inlet temperature: 250° C.
Amount of injection: 1 μl
Split ratio: 20:1
Carrier gas: He
Carrier gas flow rate: 1 ml/min
MS scan parameter
Low mass: 45
High mass: 350
Threshold: 30

1. A recombinant microorganism comprising, as foreign genes, an acyl-ACP reductase gene and a decarbonylase gene derived from blue-green algae,
provided that 4 types of blue-green algae-derived ferredoxin genes are designated as the FD gene 1, the FD gene 2, the FD gene 3, and the FD gene 4 and 3 types of blue-green algae-derived ferredoxin reductase genes are designated as the FDR gene 1, the FDR gene 2, and the FDR gene 3, the recombinant microorganism capable of synthesizing alkane comprises a ferredoxin gene and a ferredoxin reductase gene in the combination as described below:
the FD gene 1 in combination with the FDR gene 1;
the FD gene 1 in combination with the FDR gene 2;
the FD gene 2 in combination with the FDR gene 1;
the FD gene 2 in combination with the FDR gene 2;
the FD gene 2 in combination with the FDR gene 3;
the FD gene 3 in combination with the FDR gene 2
the FD gene 3 in combination with the FDR gene 3;
the FD gene 4 in combination with the FDR gene 1; or
the FD gene 4 in combination with the FDR gene 2,
wherein the FD gene 1 encodes a protein [a1] or [b1]:
[a1] a protein comprising the amino acid sequence as shown in SEQ ID NO: 2; or
[b1] a protein comprising an amino acid sequence having identity of higher than 61.2% to the amino acid sequence as shown in SEQ ID NO: 2 and functioning as ferredoxin,
wherein the FD gene 2 encodes a protein [a2] or [b2]:
[a2] a protein comprising the amino acid sequence as shown in SEQ ID NO: 4; or
[b2] a protein comprising an amino acid sequence having identity of higher than 60% to the amino acid sequence as shown in SEQ ID NO: 4 and functioning as ferredoxin,
wherein the FD gene 3 encodes a protein [a3] or [b3]:
[a3] a protein comprising the amino acid sequence as shown in SEQ ID NO: 6; or
[b3] a protein comprising an amino acid sequence having identity of higher than 62.9% to the amino acid sequence as shown in SEQ ID NO: 6 and functioning as ferredoxin,
wherein the FD gene 4 encodes a protein [a4] or [b4]:
[a4] a protein comprising the amino acid sequence as shown in SEQ ID NO: 8; or
[b4] a protein comprising an amino acid sequence having identity of higher than 62.3% to the amino acid sequence as shown in SEQ ID NO: 8 and functioning as ferredoxin,
wherein the FDR gene 1 encodes a protein [c1] or [d1]:
[c1] a protein comprising the amino acid sequence as shown in SEQ ID NO: 10; or
[d1] a protein comprising an amino acid sequence having identity of 60% or higher to the amino acid sequence as shown in SEQ ID NO: 10 and functioning as ferredoxin reductase,
wherein the FDR gene 2 encodes a protein [c2] or [d2]:
[c2] a protein comprising the amino acid sequence as shown in SEQ ID NO: 12; or
[d2] a protein comprising an amino acid sequence having identity of 60% or higher to the amino acid sequence as shown in SEQ ID NO: 12 and functioning as ferredoxin reductase, and
wherein the FDR gene 3 encodes a protein [c3] or [d3]:
[c3] a protein comprising the amino acid sequence as shown in SEQ ID NO: 14; or
[d3] a protein comprising an amino acid sequence having identity of 60% or higher to the amino acid sequence as shown in SEQ ID NO: 14 and functioning as ferredoxin reductase.

2. The recombinant microorganism according to claim 1, wherein the blue-green algae-derived decarbonylase gene encodes a protein [e] or [f]:
[e] a protein comprising the amino acid sequence as shown in SEQ ID NO: 16; or
[f] a protein comprising an amino acid sequence having identity of 60% or higher to the amino acid sequence as shown in SEQ ID NO: 16 and having decarbonylase activity.

3. The recombinant microorganism according to claim 1, wherein the blue-green algae-derived acyl-ACP reductase gene encodes a protein [g] or [h]:
[g] a protein comprising the amino acid sequence as shown in SEQ ID NO: 18; or
[h] a protein comprising an amino acid sequence having identity of 60% or higher to the amino acid sequence as shown in SEQ ID NO: 18 and having acyl ACP reductase activity.

4. The recombinant microorganism according to claim 1, wherein host cells are E. coli or Klebsiella bacteria.

5. A method for producing alkane comprising a step of culturing the recombinant microorganism according to claim 1.

6. The method for producing alkane according to claim 5, which further comprises a step of recovering alkane from a medium in which the recombinant microorganism is cultured.

7. The method for producing alkane according to claim 5, which further comprises a step of recovering alkane from a medium in which the recombinant microorganism is cultured and purifying the recovered alkane.

8. The method for producing alkane according to claim 5, wherein alkane having 9 to 20 carbon atoms is produced.


SEQUENCE LISTING (58 sequences)

SEQ ID NO: 1 - DNA, 300 bp, Nostoc punctiforme PCC 73102
  CDS (1)..(300)
atggctacctaccaagttagattaatcaacaaaaaagaagacatcgacaccacaattgaggttgatgaagaaactaccatcttagaagcagcagaagaaaatggtattgaattgcctttttcatgtcatgcgggatcttgctctagctgtgtaggcaaggttgttgaaggcgaagttaatcaagaagatcaaaacttcctggatgacgatcaggtttctaaaggatacgctctactttgtgtaacttatcctcgttctaattgcacaatcaaaacacatcaagaagcatatctcgtataa

SEQ ID NO: 2 - protein, 99 aa, Nostoc punctiforme PCC 73102
MATYQVRLINKKEDIDTTIEVDEETTILEAAEENGIELPFSCHAGSCSSCVGKVVEGEVNQEDQNFLDDDQVSKGYALLCVTYPRSNCTIKTHQEAYLV

SEQ ID NO: 3 - DNA, 369 bp, Nostoc punctiforme PCC 73102
  CDS (1)..(369)
atgtcccgtacatacacaattaaagttcgcgatcgcgccactggcaaaacacacaccctaaaagtgccagaagaccgttatatcctgcacactgccgaaaaacaaggtgtggaactaccgttttcctgtcgcaacggagcttgcaccgcttgtgctgtgagggtattgtcaggagaaatttatcaaccagaggcgatcggattgtcaccagatttacgtcagcaaggttatgccctgttgtgtgtgagttatccccgttctgacttggaagtagagacacaagacgaagatgaagtctacgaactccagtttgggcgctattttgctaaggggaaagttaaagcgggtttaccgttagatgaggaataa

SEQ ID NO: 4 - protein, 122 aa, Nostoc punctiforme PCC 73102
MSRTYTIKVRDRATGKTHTLKVPEDRYILHTAEKQGVELPFSCRNGACTACAVRVLSGEIYQPEAIGLSPDLRQQGYALLCVSYPRSDLEVETQDEDEVYELQFGRYFAKGKVKAGLPLDEE

SEQ ID NO: 5 - DNA, 321 bp, Nostoc punctiforme PCC 73102
  CDS (1)..(321)
atgcccaaaacttacaccgtagaaatcgatcatcaaggcaaaattcataccttgcaagttcctgaaaatgaaacgatcttatcagttgccgatgctgctggtttggaactgccgagttcttgtaatgcaggtgtttgcacaacttgcgccggtcaaataagccagggaactgtggatcaaactgatggcatgggcgttagtccagatttacaaaagcaaggttacgtattgctttgtgttgcgaaacccctttctgatttgaaacttgaaacagaaaaggaagacatagtttatcagttacaatttggcaaagacaaataa

SEQ ID NO: 6 - protein, 106 aa, Nostoc punctiforme PCC 73102
MPKTYTVEIDHQGKIHTLQVPENETILSVADAAGLELPSSCNAGVCTTCAGQISQGTVDQTDGMGVSPDLQKQGYVLLCVAKPLSDLKLETEKEDIVYQLQFGKDK

SEQ ID NO: 7 - DNA, 300 bp, Nostoc punctiforme PCC 73102
  CDS (1)..(300)
atgccaacttataaagtgacactaattaacgaggctgaagggctgaacacaacccttgatgttgaggacgatacctatattctagacgcagctgaagaagctggtattgacctgccctactcttgccgcgctggtgcttgctctacttgtgcaggtaaactcgtatcaggtaccgtcgatcaaggcgatcaatcattcttagatgacgatcaaatagaagctggatatgtactgacctgtgttgcttacccaacttctaatgtcacgatcgaaactcacaaagaagaagaactctattaa

SEQ ID NO: 8 - protein, 99 aa, Nostoc punctiforme PCC 73102
MPTYKVTLINEAEGLNTTLDVEDDTYILDAAEEAGIDLPYSCRAGACSTCAGKLVSGTVDQGDQSFLDDDQIEAGYVLTCVAYPTSNVTIETHKEEELY

SEQ ID NO: 9 - DNA, 1371 bp, Nostoc punctiforme PCC 73102
  CDS (1)..(1371)
atgaacagcccagtttatcaaactgtgattgtcggcggtggttttaccgggctatttacagccttacatttagctcacgaacattatccccgctctgttatcttgattgataaagacgagcgcttttgctttaagccactattatatgaatatttcgatggtgagatggatacctttcaggtagtgccgcgtttttcggaactgcttaaaggtagtggtgtcatctttgtgcaagatacagtgcaatcgataaacttgcatcaacgggaagttaaattagcttcgggaaattcctacaattacagcaatttagtattagctttgggcagcgttactggctatcatcaagttgaaggtgctagagaaaatgcctttcctttctggacgcaagcagatgcgatcgctcttgaccgtcatttacgtgactgtttgcaaaaagctgtccaaactgaagatgtagaacaacgccgaaagctattaacagtagtcatagttggtggtggtgcaagtggtgtggaaatggcagcaactctagctgattttctcccacattggtatagcgctttgggaggaaattcaagtgaaattcgtgtagtactactcaatcatggtaaagaaatccttgatggtgatataaataacccattgcgtgaaattgccgaacgggaattgaacaaacgtgctgtgccaatagaaatgattgtggaagcagaagctactgctattggttccacctcaattcaatataagcgtaacgagcaaattgagacattagctacacatactacagtttggactgctggcacttctacccatccactgattaaagatttgccaattcctaaagaacatcgagatcgtcgcgatcgccctttagttactcctaacttgcaattactcgattttccagaagtctttgtcggtggtgattgtgcagcagttcaggataattcgctaccgcctacggctcaagttgcttatcagcaaggagccaatattgcccagaatttgaaagcattagcattaggagaagaacccaaacctgctaaggttaacatccggggaacccttttgaaattggggataaatgatgctgctgctaaccttttcaatgtttttgaagttgtaggcgaaaccgcgcatttaatccgtcaaggcacttatttaacgctattaccagcaccaattcatgactttaaagccaccactgaatgggtagatgaagaagtttttcatcagcaccttgaccctcatgatgtcgggaaaaaagtggtgcaagcagcggaaatagtcggcgtaggagttgtaggtattctagctgcaagaaaattactgaaaatgttgggagatgaagataaaaacagtaagtag

SEQ ID NO: 10 - protein, 456 aa, Nostoc punctiforme PCC 73102
MNSPVYQTVIVGGGFTGLFTALHLAHEHYPRSVILIDKDERFCFKPLLYEYFDGEMDTFQVVPRFSELLKGSGVIFVQDTVQSINLHQREVKLASGNSYNYSNLVLALGSVTGYHQVEGARENAFPFWTQADAIALDRHLRDCLQKAVQTEDVEQRRKLLTVVIVGGGASGVEMAATLADFLPHWYSALGGNSSEIRVVLLNHGKEILDGDINNPLREIAERELNKRAVPIEMIVEAEATAIGSTSIQYKRNEQIETLATHTTVWTAGTSTHPLIKDLPIPKEHRDRRDRPLVTPNLQLLDFPEVFVGGDCAAVQDNSLPPTAQVAYQQGANIAQNLKALALGEEPKPAKVNIRGTLLKLGINDAAANLFNVFEVVGETAHLIRQGTYLTLLPAPIHDFKATTEWVDEEVFHQHLDPHDVGKKVVQAAEIVGVGVVGILAARKLLKMLGDEDKNSK

SEQ ID NO: 11 - DNA, 1314 bp, Nostoc punctiforme PCC 73102
  CDS (1)..(1314)
atgtacaatcaaggtgctgttgagggtgctgccaacatagaattaggtagccgcatcttcgtttatgaagtagtgggtttgcgtcagggggaagaaaccgatcaaactaactacccaattcggaaaagtggcagtgtgttcatcagagtgccttacaaccgcatgaatcaagaaatgcgacgtatcactcgtctaggcggcacaattgttagcatccaacctataactgctctagaaccagttaatggtaaagcctcatttgggaatgctacaagcgttgtcagcgaattagctaaatctggggaaactgctaacagtgaagggaatggtaaagccacacctgtaaatgctcatagtgctgaagaacagaacaaggacaagaaaggcaacaccatgactcaagcgaaagccaaaaaagaccacggtgacgttcctgttaacacttaccgtcccaatgctccatttattggcaaggtaatatctaatgaaccattagtcaaagaaggtggtattggtattgttcaacaccttaaatttgacctatctggtggggatttgaagtatatagaaggtcaaagtattggcattattccgccaggtttagacaagaacggcaagcctgaaaaactcagactatattccatcgcctcaactcgtcatggtgatgatgtagatgataagacagtatcactgtgcgtccgccagttggagtacaagcacccagaaactggcgaaacagtctacggtgtttgctctacgcacctgtgtttcctcaagccaggggaagaggtaaaaattacagggcctgtgggtaaggaaatgttgttacccaatgaccctgatgctaatgttatcatgatggctactggaacaggtattgcgccgatgcgggcttacttgtggcgtcagtttaaagatgcggaaagagcggctaacccagaataccaatttaaaggattctcttggctaatatttggcgtacctacaactccaaaccttttatataaggaagaactggaagagattcaacaaaaatatcctgagaacttccgcctaactgctgccatcagccgcgaacagaaaaatccccaaggcggtagaatgtatattcaagaccgcgtagcagaacatgctgatgaattgtggcagttgattaaaaatgaaaaaacccacacttacatttgcggtttgcgcggtatggaagaaggtattgatgcagccttaactgctgctgctgctaaggaaggcgtaacctggagtgattaccagaagcaactcaagaaagccggtcgctggcacgtagaaacttactaa

SEQ ID NO: 12 - protein, 437 aa, Nostoc punctiforme PCC 73102
MYNQGAVEGAANIELGSRIFVYEVVGLRQGEETDQTNYPIRKSGSVFIRVPYNRMNQEMRRITRLGGTIVSIQPITALEPVNGKASFGNATSVVSELAKSGETANSEGNGKATPVNAHSAEEQNKDKKGNTMTQAKAKKDHGDVPVNTYRPNAPFIGKVISNEPLVKEGGIGIVQHLKFDLSGGDLKYIEGQSIGIIPPGLDKNGKPEKLRLYSIASTRHGDDVDDKTVSLCVRQLEYKHPETGETVYGVCSTHLCFLKPGEEVKITGPVGKEMLLPNDPDANVIMMATGTGIAPMRAYLWRQFKDAERAANPEYQFKGFSWLIFGVPTTPNLLYKEELEEIQQKYPENFRLTAAISREQKNPQGGRMYIQDRVAEHADELWQLIKNEKTHTYICGLRGMEEGIDAALTAAAAKEGVTWSDYQKQLKKAGRWHVETY

SEQ ID NO: 13 - DNA, 738 bp, Nostoc punctiforme PCC 73102
  CDS (1)..(738)
atgtcatttacttctatgccctcgctgcgtgagcaacaacatcccctaattcgtcagctagctgattgtattgaggcagcttggcatcagcacctggatctatcgccctaccatttgcctgatgagttggggtatgtggaaggtagactagaaggcgaaaaactgacgattgaaaatcggtgctatcaaacgccccagtttcgcaaaatgcacttggaactggcaaacatcggaaatatgctggatattttgcactgcgtcatgtttccccgtccccaatacaacctgccgatgtttggttgtgatttagttgggggtagaggtcaaattagtgcagcgatcgcagatctttcccctatccaattagagcgcaccttaccagaatcttatactactgcactggcacagctaccagtgcttaacttttcccaaccccgtgaattaccagaatggggaaatattttttcggatttttgcatctttgtgcgtcccggttccccagaagaagaagcaatgtttctgtcgcgtgtgcgagaatttttagacattcattgtatgcaagcgatcgcttcacatcctgtttcagtcgaacaagttacacaaaatctcgctggacaacacaactactgtactaaacagcagcaaaacgataaaacccgccgcgtactagaaaaagcctttggtccagtttgggcagaaaattatatgaccacagttttattcgaccttccaacttaa

SEQ ID NO: 14 - protein, 245 aa, Nostoc punctiforme PCC 73102
MSFTSMPSLREQQHPLIRQLADCIEAAWHQHLDLSPYHLPDELGYVEGRLEGEKLTIENRCYQTPQFRKMHLELANIGNMLDILHCVMFPRPQYNLPMFGCDLVGGRGQISAAIADLSPIQLERTLPESYTTALAQLPVLNFSQPRELPEWGNIFSDFCIFVRPGSPEEEAMFLSRVREFLDIHCMQAIASHPVSVEQVTQNLAGQHNYCTKQQQNDKTRRVLEKAFGPVWAENYMTTVLFDLPT

SEQ ID NO: 15 - DNA, 699 bp, Nostoc punctiforme PCC 73102
  CDS (1)..(699)
atgcagcagcttacagaccaatctaaagaattagatttcaagagcgaaacatacaaagatgcttatagccggattaatgcgatcgtgattgaaggggaacaagaagcccatgaaaattacatcacactagcccaactgctgccagaatctcatgatgaattgattcgcctatccaagatggaaagccgccataagaaaggatttgaagcttgtgggcgcaatttagctgttaccccagatttgcaatttgccaaagagtttttctccggcctacaccaaaattttcaaacagctgccgcagaagggaaagtggttacttgtctgttgattcagtctttaattattgaatgttttgcgatcgcagcatataacatttacatccccgttgccgacgatttcgcccgtaaaattactgaaggagtagttaaagaagaatacagccacctcaattttggagaagtttggttgaaagaacactttgcagaatccaaagctgaacttgaacttgcaaatcgccagaacctacccatcgtctggaaaatgctcaaccaagtagaaggtgatgcccacacaatggcaatggaaaaagatgctttggtagaagacttcatgattcagtatggtgaagcattgagtaacattggtttttcgactcgcgatattatgcgcttgtcagcctacggactcataggtgcttaa

SEQ ID NO: 16 - protein, 232 aa, Nostoc punctiforme PCC 73102
MQQLTDQSKELDFKSETYKDAYSRINAIVIEGEQEAHENYITLAQLLPESHDELIRLSKMESRHKKGFEACGRNLAVTPDLQFAKEFFSGLHQNFQTAAAEGKVVTCLLIQSLIIECFAIAAYNIYIPVADDFARKITEGVVKEEYSHLNFGEVWLKEHFAESKAELELANRQNLPIVWKMLNQVEGDAHTMAMEKDALVEDFMIQYGEALSNIGFSTRDIMRLSAYGLIGA

SEQ ID NO: 17 - DNA, 1026 bp, Synechococcus elongatus PCC7942
  CDS (1)..(1026)
atgttcggtcttatcggtcatctcaccagtttggagcaggcccgcgacgtttctcgcaggatgggctacgacgaatacgccgatcaaggattggagttttggagtagcgctcctcctcaaatcgttgatgaaatcacagtcaccagtgccacaggcaaggtgattcacggtcgctacatcgaatcgtgtttcttgccggaaatgctggcggcgcgccgcttcaaaacagccacgcgcaaagttctcaatgccatgtcccatgcccaaaaacacggcatcgacatctcggccttggggggctttacctcgattattttcgagaatttcgatttggccagtttgcggcaagtgcgcgacactaccttggagtttgaacggttcaccaccggcaatactcacacggcctacgtaatctgtagacaggtggaagccgctgctaaaacgctgggcatcgacattacccaagcgacagtagcggttgtcggcgcgactggcgatatcggtagcgctgtctgccgctggctcgacctcaaactgggtgtcggtgatttgatcctgacggcgcgcaatcaggagcgtttggataacctgcaggctgaactcggccggggcaagattctgcccttggaagccgctctgccggaagctgactttatcgtgtgggtcgccagtatgcctcagggcgtagtgatcgacccagcaaccctgaagcaaccctgcgtcctaatcgacgggggctaccccaaaaacttgggcagcaaagtccaaggtgagggcatctatgtcctcaatggcggggtagttgaacattgcttcgacatcgactggcagatcatgtccgctgcagagatggcgcggcccgagcgccagatgtttgcctgctttgccgaggcgatgctcttggaatttgaaggctggcatactaacttctcctggggccgcaaccaaatcacgatcgagaagatggaagcgatcggtgaggcatcggtgcgccacggcttccaacccttggcattggcaatttga

SEQ ID NO: 18 - protein, 341 aa, Synechococcus elongatus PCC7942
MFGLIGHLTSLEQARDVSRRMGYDEYADQGLEFWSSAPPQIVDEITVTSATGKVIHGRYIESCFLPEMLAARRFKTATRKVLNAMSHAQKHGIDISALGGFTSIIFENFDLASLRQVRDTTLEFERFTTGNTHTAYVICRQVEAAAKTLGIDITQATVAVVGATGDIGSAVCRWLDLKLGVGDLILTARNQERLDNLQAELGRGKILPLEAALPEADFIVWVASMPQGVVIDPATLKQPCVLIDGGYPKNLGSKVQGEGIYVLNGGVVEHCFDIDWQIMSAAEMARPERQMFACFAEAMLLEFEGWHTNFSWGRNQITIEKMEAIGEASVRHGFQPLALAI

SEQ ID NO: 19 - DNA, 294 bp, Nostoc punctiforme PCC 73102
  CDS (1)..(294)
atggcacaactaacaggtttgacatttggcggtaaagcttggacaccaaaatttgctcaagaaattgacaaagataaatgtatcggctgtggcagatgtgttaaagtctgcgggtacaatgtgctaggtttgaaggcactcaatgaagaaggtgaatttgtagaagacgaagatgatgaagaaattgaacggaaagtaatggcagttacttctccagaaaactgtattggttgtgaagcgtgttcgcggatttgcccgaaaaattgctacacccatgttgcagtaaataattag

SEQ ID NO: 20 - protein, 97 aa, Nostoc punctiforme PCC 73102
MAQLTGLTFGGKAWTPKFAQEIDKDKCIGCGRCVKVCGYNVLGLKALNEEGEFVEDEDDEEIERKVMAVTSPENCIGCEACSRICPKNCYTHVAVNN

SEQ ID NO: 21 - DNA, 1371 bp, Artificial
  Synthetic DNA
atgaactctcctgtctatcaaactgtcattgtcggtggtggttttactggtttattcactgctttacacttggctcacgaacattatccaagatccgttattttgatcgataaggacgaaagattctgtttcaagcctttgttgtacgaatacttcgatggtgaaatggacacatttcaagttgtcccaagattctccgaattgttaaaaggtagtggtgtcatattcgtacaagataccgtacaatctatcaatttgcatcaaagagaggttaagttggcatctggtaactcatacaactactctaatttggtcttggctttgggttcagtaaccggttatcatcaagttgaaggtgctagagaaaatgcatttccattctggactcaagctgatgcaattgccttagacagacacttgagagattgcttacaaaaagcagtacaaactgaagatgttgaacaaagaagaaagttgttgacagtagttatagttggtggtggtgcttccggtgtcgaaatggctgcaacattggccgatttcttgcctcattggtatagtgctttaggtggtaactcttcagaaatcagagtcgtattgttgaaccacggtaaagaaatcttggatggtgacatcaacaacccattaagagaaatcgcagaaagagaattgaacaagagagccgtccctatcgaaatgattgtagaagctgaagcaacagccattggttccaccagtatccaatacaagagaaacgaacaaatcgaaactttggctacacatactacagtttggaccgcaggtacatctacccacccattgatcaaagacttaccaattcctaaggaacatagagatagaagagacagaccattggttacacctaacttacaattgttggatttccctgaagttttcgtcggtggtgactgtgccgctgtccaagataattcattaccacctactgcccaagttgcttatcaacaaggtgcaaacattgcccaaaatttgaaagctttagcattgggtgaagaaccaaaacctgctaaggttaacatcagaggtactttgttgaagttgggtataaatgatgcagccgctaacttgtttaatgttttcgaagttgtcggtgaaacagctcatttgatcagacaaggtacctacttaactttgttaccagcccctattcacgactttaaagctaccactgaatgggttgatgaagaagtcttccatcaacacttggatccacatgacgttggtaaaaaggtagttcaagcagccgaaattgtaggtgttggtgtcgtaggtatattagccgccagaaaattgttgaagatgttaggtgacgaagataaaaactccaaataa

SEQ ID NO: 22 - DNA, 1314 bp, Artificial
  Synthetic DNA
atgtataatcaaggtgccgtcgaaggtgctgctaacatagaattgggtagtagaatctttgtttacgaagtagtaggtttaagacaaggtgaagaaaccgatcaaactaattatccaatcagaaagtctggttcagtttttattagagtcccttacaacagaatgaaccaagaaatgagaagaataactagattgggtggtacaatagtatccatccaaccaattaccgctttagaacctgttaatggtaaagctagtttcggtaacgcaacatccgttgtcagtgaattggccaagtccggtgaaaccgctaatagtgagggtaacggtaaagcaactccagttaatgctcattcagcagaagaacaaaataaggataaaaagggtaacaccatgactcaagcaaaagccaaaaaggatcacggtgacgtaccagttaatacttatagaccaaacgctccttttataggtaaagttatttctaacgaacctttggtcaaagaaggtggtatcggtattgttcaacatttgaagttcgatttgtctggtggtgacttgaagtacatcgaaggtcaatcaatcggtataataccacctggtttggataagaacggtaaaccagaaaagttgagattgtactctatagcatcaacaagacatggtgacgacgttgatgacaagaccgtatctttgtgtgttagacaattggaatacaagcatccagaaacaggtgaaaccgtctacggtgtatgttcaactcacttgtgctttttaaagccaggtgaagaagtcaaaattacaggtcctgttggtaaagaaatgttgttaccaaatgatcctgacgcaaacgtcataatgatggccacaggtaccggtatcgcaccaatgagagcctatttgtggagacaattcaaagatgctgaaagagctgcaaatcctgaataccaattcaaaggtttctcctggttaattttcggtgttccaactacacctaacttgttgtacaaggaagaattggaagaaatccaacaaaagtacccagaaaacttcagattgactgccgctatttctagagaacaaaagaatcctcaaggtggtagaatgtatatacaagatagagttgccgaacatgctgacgaattgtggcaattaattaagaatgaaaagactcacacatacatatgcggtttgcgtggtatggaagaaggtattgatgcagccttaacagctgcagccgctaaagaaggtgttacttggagtgactatcaaaagcaattaaagaaggcaggtagatggcacgtagaaacttattaa

SEQ ID NO: 23 - DNA, 738 bp, Artificial
  Synthetic DNA
atgagtttcaccagtatgccatctttgagagaacaacaacaccctttgattagacaattagcagactgtatcgaagccgcttggcaccaacatttggatttgtctccatatcacttacctgacgaattgggttacgttgaaggtagattggaaggtgaaaagttgaccatcgaaaacagatgttaccaaactccacaattcagaaagatgcatttggaattagccaatatcggtaacatgttggatattttacactgtgtcatgttcccaagacctcaatacaacttacctatgtttggttgcgatttggttggtggtagaggtcaaatttccgctgcaatagcagacttaagtccaatacaattggaaagaactttgcctgaatcatacactacagccttggctcaattaccagttttgaatttttcccaaccaagagaattgcctgaatggggtaacattttctctgatttctgcatctttgtaagaccaggttctcctgaagaagaagctatgttcttgtcaagagttagagaattcttggacatccattgtatgcaagcaatagcctctcacccagtttcagtcgaacaagttacacaaaatttggcaggtcaacataactactgcacaaaacaacaacaaaatgataagaccagaagagtcttggaaaaagccttcggtcctgtctgggctgaaaactacatgactactgtcttatttgacttgcctacttaa

SEQ ID NO: 24 - DNA, 294 bp, Artificial
  Synthetic DNA
atggcacaattgactggtttgacattcggtggtaaagcctggacccctaagttcgcccaagaaatagacaaggacaagtgtatcggttgtggtagatgcgttaaagtctgtggttataatgtcttgggtttaaaggctttgaacgaagagggtgaatttgttgaagatgaagatgacgaagaaatcgaaagaaaagtaatggcagttacttctccagaaaattgcataggttgtgaagcctgctcaagaatctgtccaaagaactgctacacccatgttgccgtcaataactaa

SEQ ID NO: 25 - DNA, 300 bp, Artificial
  Synthetic DNA
atggcaacctaccaagtcagattgattaacaaaaaggaagacatcgacacaactattgaagtagacgaagaaaccactatattagaagctgcagaagaaaatggtattgaattgccattttcctgtcatgccggtagttgttcttcatgcgttggtaaagttgtcgaaggtgaagttaatcaagaagaccaaaacttcttagatgacgatcaagtctctaagggttatgctttgttatgcgtcacttacccaagatccaactgcaccatcaaaacccaccaagaagcatacttagtctaa

SEQ ID NO: 26 - DNA, 369 bp, Artificial
  Synthetic DNA
atgagtagaacatacaccataaaagttagagatagagccacaggtaaaacacacaccttgaaagtcccagaagatagatacatattgcatactgcagaaaaacaaggtgtcgaattgccattttcttgtagaaatggtgcttgtacagcttgcgcagttagagtcttatccggtgaaatctatcaaccagaagccataggtttgagtcctgatttgagacaacaaggttatgctttgttatgcgtttcttaccctagatcagacttggaagtagaaacccaagatgaagacgaagtttacgaattacaattcggtagatatttcgccaagggtaaagtaaaagcaggtttgccattagatgaagaataa

SEQ ID NO: 27 - DNA, 321 bp, Artificial
  Synthetic DNA
atgcctaaaacatacaccgttgaaatagaccaccaaggtaaaatacacaccttacaagtcccagaaaatgaaacaatcttatcagtagcagatgctgcaggtttggaattaccatcttcatgtaatgccggtgtctgtactacatgcgctggtcaaatttcccaaggtaccgtagatcaaactgacggtatgggtgttagtccagatttgcaaaagcaaggttacgttttgttgtgcgtcgctaagcctttatctgacttaaagttagaaacagaaaaagaagacatagtatatcaattacaattcggtaaagacaagtaa

SEQ ID NO: 28 - DNA, 300 bp, Artificial
  Synthetic DNA
atgccaacatacaaggtcactttgattaacgaagcagaaggtttgaatacaactttagacgtagaagacgatacttacattttagacgctgcagaagaagcaggtattgacttgccatattcttgtagagccggtgcttgttcaacttgcgccggtaaattagtttccggtacagtcgaccaaggtgaccaatcatttttggatgacgatcaaatagaagctggttacgttttaacctgtgttgcctaccctaccagtaatgtcactattgaaacccataaggaagaagaattgtattaa

SEQ ID NO: 29 - DNA, 340 bp, Artificial
  Synthetic DNA
ataccatggaaccgaaaacctataccgtggaaattgatcatcagggcaaaatccataccctgcaggtgccggaaaacgaaaccattctgagcgttgccgatgccgcgggcctggaactgccgagcagctgcaatgcgggtgtttgcaccacctgtgccggccagatcagccagggtaccgtggatcagaccgatggcatgggtgttagcccggatctgcagaaacagggctatgtgctgctgtgtgttgccaaaccgctgagcgatctgaaactggaaaccgaaaaagaagatattgtgtatcagctgcagtttggtaaagataaataagcggccgcata

SEQ ID NO: 30 - DNA, 1332 bp, Artificial
  Synthetic DNA
atacatatggaatataatcagggtgccgtggaaggtgccgcgaacattgaactgggtagccgtatttttgtctatgaagtggtcggtctgcgtcagggtgaagaaaccgatcagaccaactatccgatccgtaaaagcggcagcgtgtttattcgtgttccgtataaccgcatgaatcaggaaatgcgtcgcattacccgcctgggcggtaccattgtgagcatccagccgattaccgcgctggaaccggttaacggcaaagcgagctttggtaatgccaccagcgtggttagcgaactggcgaaaagcggtgaaaccgccaacagcgaaggcaatggtaaagccaccccggtgaatgcgcatagcgccgaagaacagaacaaagataagaaaggcaataccatgacccaggcgaaagcgaagaaagatcatggcgatgtgccggttaacacctatcgtccgaatgcgccgtttatcggcaaagtgattagcaacgaaccgctggttaaagaaggcggtattggtatcgtgcagcatctgaaatttgatctgagcggcggtgatctgaaatatatcgaaggccagagcattggtattatcccgccgggcctggataaaaatggtaaaccggaaaaactgcgtctgtatagcattgcgagcacccgtcatggcgatgatgttgatgataaaaccgtgagcctgtgcgttcgtcagctggaatataaacatccggaaaccggcgaaaccgtgtatggtgtttgcagcacccatctgtgttttctgaaaccgggtgaagaagtgaaaatcaccggcccggttggtaaagaaatgctgctgccgaacgatccggatgcgaatgtgatcatgatggcaaccggtaccggtattgccccgatgcgtgcctatctgtggcgccagtttaaagatgcggaacgcgcggccaacccggaatatcagtttaaaggctttagctggctgatttttggtgttccgaccaccccgaatctgctgtataaagaagaactggaagaaatccagcagaaatatccggaaaactttcgtctgaccgcggccattagccgcgaacagaaaaatccgcagggcggtcgtatgtatatccaggatcgcgtggcggaacatgccgatgaactgtggcagctgatcaaaaacgaaaaaacccatacctatatttgtggcctgcgcggtatggaagaaggcattgatgccgcactgaccgcggcggcggcaaaagaaggtgttacctggtcggattatcaaaaacaactgaaaaaggctggtcgttggcatgtggaaacctattaactcgagtct

SEQ ID NO: 31 - DNA, 36 bp, Artificial
  Synthetic DNA
aaggagatatacatatgaactctcctgtctatcaaa

SEQ ID NO: 32 - DNA, 39 bp, Artificial
  Synthetic DNA
cagcagcctaggttattatttggagtttttatcttcgtc

SEQ ID NO: 33 - DNA, 36 bp, Artificial
  Synthetic DNA
aaggagatatacatatgtataatcaaggtgccgtcg

SEQ ID NO: 34 - DNA, 38 bp, Artificial
  Synthetic DNA
cagcagcctaggttattaataagtttctacgtgccatc

SEQ ID NO: 35 - DNA, 34 bp, Artificial
  Synthetic DNA
aaggagatatacatatgagtttcaccagtatgcc

SEQ ID NO: 36 - DNA, 37 bp, Artificial
  Synthetic DNA
cagcagcctaggttattaagtaggcaagtcaaataag

SEQ ID NO: 37 - DNA, 33 bp, Artificial
  Synthetic DNA
aggagatataccatggcacaattgactggtttg

SEQ ID NO: 38 - DNA, 36 bp, Artificial
  Synthetic DNA
aagcattatgcggccttagttattgacggcaacatg

SEQ ID NO: 39 - DNA, 32 bp, Artificial
  Synthetic DNA
aggagatataccatggcaacctaccaagtcag

SEQ ID NO: 40 - DNA, 36 bp, Artificial
  Synthetic DNA
aagcattatgcggccttagactaagtatgcttcttg

SEQ ID NO: 41 - DNA, 33 bp, Artificial
  Synthetic DNA
aggagatataccatgagtagaacatacaccata

SEQ ID NO: 42 - DNA, 38 bp, Artificial
  Synthetic DNA
aagcattatgcggccttattcttcatctaatggcaaac

SEQ ID NO: 43 - DNA, 34 bp, Artificial
  Synthetic DNA
aggagatataccatgcctaaaacatacaccgttg

SEQ ID NO: 44 - DNA, 36 bp, Artificial
  Synthetic DNA
aagcattatgcggccttacttgtctttaccgaattg

SEQ ID NO: 45 - DNA, 32 bp, Artificial
  Synthetic DNA
aggagatataccatgccaacatacaaggtcac

SEQ ID NO: 46 - DNA, 39 bp, Artificial
  Synthetic DNA
aagcattatgcggccttaatacaattcttcttccttatg

SEQ ID NO: 47 - DNA, 36 bp, Artificial
  Synthetic DNA
aggaaacagaccatggaaccgaaaacctataccgtg

SEQ ID NO: 48 - DNA, 40 bp, Artificial
  Synthetic DNA
gaaattgttatccgcggccgcttatttatctttaccaaac

SEQ ID NO: 49 - DNA, 51 bp, Artificial
  Synthetic DNA
cggataacaatttcacacaggaaacagacatggaatataatcagggtgccg

SEQ ID NO: 50 - DNA, 39 bp, Artificial
  Synthetic DNA
gtaatcatggccatgttaataggtttccacatgccaacg

SEQ ID NO: 51 - DNA, 40 bp, Artificial
  Synthetic DNA
cgagctcggcgcgcctgcagatgcagcagcttacagacca

SEQ ID NO: 52 - DNA, 40 bp, Artificial
  Synthetic DNA
gcaagcttgtcgacctgcagttaagcacctatgagtccgt

SEQ ID NO: 53 - DNA, 37 bp, Artificial
  Synthetic DNA
aaggagatatacatatgatgttcggtcttatcggtca

SEQ ID NO: 54 - DNA, 37 bp, Artificial
  Synthetic DNA
ttgagatctgccatatgtcaaattgccaatgccaagg

SEQ ID NO: 55 - DNA, 38 bp, Artificial
  Synthetic DNA
aggaaacagacgtacatgcagcagcttacagaccaatc

SEQ ID NO: 56 - DNA, 37 bp, Artificial
  Synthetic DNA
gaaattgttatccgcttaagcacctatgagtccgtag

SEQ ID NO: 57 - DNA, 52 bp, Artificial
  Synthetic DNA
gcggataacaatttcacacaggaaacagacatgttcggtcttatcggtcatc

SEQ ID NO: 58 - DNA, 35 bp, Artificial
  Synthetic DNA
gtaatcatggcgtactcaaattgccaatgccaagg